(19) United States
(12) Patent Application Publication
(10) Pub. No.: US 2017/0051311 A1
(43) Pub. Date: Feb. 23, 2017

(54) METHODS AND APPARATUS FOR TRANSFORMATION OF NATURALLY COMPETENT CELLS

(71) Applicant: TUFTS UNIVERSITY, MEDFORD, MA (US)

(72) Inventors: ANKUR B. DALIA, BOSTON, MA (US); ANDREW CAMILLI, BOSTON, MA (US)

(73) Assignee: TUFTS UNIVERSITY, MEDFORD, MA (US)

(21) Appl. No.: 15/308,329

(22) PCT Filed: May 1, 2015
(86) PCT No.: PCT/US15/28851

§ 371 Date: Nov. 1, 2016

Related U.S. Application Data
(60) Provisional application No. 61/987,955, filed on May 2, 2014.

(51) Int. Cl. C12N 15/90

(52) U.S. Cl. CPC C12N 15/902

(57) ABSTRACT

The present invention includes compositions and methods of co-transformation of naturally competent cells. In one aspect of the invention, a method is included for introducing nucleic acid sequences into one or more naturally competent cells in parallel. In other aspects, a heterogenic pool of co-transformed naturally competent cells and an apparatus for introducing two or more populations of nucleic acid sequences into a population of naturally competent cells in parallel are also included.

### CROSS REFERENCE TO RELATED APPLICATIONS

This application claims benefit of U.S. Provisional Application Ser. No. 61/987,955, filed on May 2, 2014, the contents of which are incorporated herein by reference.

### STATEMENT REGARDING FEDERALLY SPONSORED RESEARCH OR DEVELOPMENT

This invention was made with government support under AI055058 and AI045746, awarded by the National Institutes of Health. The government has certain rights in the invention.

### BACKGROUND OF THE INVENTION

Tools for multiplexed genome editing, i.e., simultaneous editing at multiple distinct sites in a genome, are limited in number and currently only developed for use in model bacteria. The method known as “multiplexed automated genome engineering” or MAGE was developed in Escherichia coli, and has been widely successful in “accelerated evolution” of this species, which has been exploited for metabolic and phenotypic engineering applications. This technique was also critical for “recoding” the E. coli genome, in which all UAG stop codons were replaced with synonymous UAA codons.

MAGE relies on highly efficient recombineering with single-stranded DNA (ssDNA) oligonucleotides. Mechanistically this method requires annealing of ssDNA oligos to the lagging strand during DNA replication and can introduce point mutations or small insertions and deletions into the genome at efficiencies of up to ˜20%. A key feature of this technique is the absence of selection for mutations in cis, which allows for multiplexed mutations to be randomly distributed in output mutant pools, where individual cells in this population have any number and combination of genome edits. MAGE demonstrates the utility of methods for multiplexed genome editing in microbial systems, however, this method is not easily adapted to non-model microorganisms.

Recently, the Cas-9 endonuclease derived from the bacterial CRISPR/Cas system, has been exploited for targeted genome engineering in non-model bacterial microorganisms. This method, however, requires Cas9 selection at edited genomic loci. Therefore, CRISPR/Cas-mediated genome editing cannot produce complex mutant pools as described above for MAGE, and limits the use of this technique for accelerated evolution of phenotypes in microbial systems.

Therefore, a need exists in the art for improved methods for multiplex genome editing in microbial systems that are non-model microorganisms.

### SUMMARY OF THE INVENTION

The invention generally features methods for transforming a naturally competent micro-organism simultaneously with two or more nucleic acid molecules and cells comprising these molecules.

In one aspect, the invention generally provides a method for introducing nucleic acid molecules into one or more naturally competent cells in parallel. In other aspects, a method of introducing nucleic acid molecules into one or more polynucleotide targets in parallel and a method for optimizing the transformation efficiency of a naturally competent cell are included. In other aspects, a heterogenic pool of co-transformed naturally competent cells and an apparatus for introducing two or more populations of nucleic acid molecules into a population of naturally competent cells in parallel are also included.

In one aspect, the invention includes a method of introducing nucleic acid molecules into one or more cells in parallel comprising: (a) contacting naturally competent cells with two or more nucleic acid molecules, wherein at least one of the nucleic acid sequences comprises a selectable marker; and (b) selecting for that marker.

In another aspect, the invention includes a method of introducing nucleic acid molecules into one or more cells in parallel comprising: (a) incubating naturally competent cells under static conditions; (b) contacting the cells with two or more nucleic acid molecules, wherein at least one of the nucleic acid sequences comprises a selectable marker; and (c) selecting for that marker.

In another aspect, the invention includes a method of introducing nucleic acid molecules into one or more polynucleotide targets in parallel comprising: (a) contacting the polynucleotide target with two or more nucleic acid molecules, wherein at least one of the nucleic acid sequences comprises a selectable marker; and (b) selecting for that marker.

In another aspect, the invention includes a method for optimizing the transformation efficiency of a naturally competent cell, the method comprising introducing a genetic mutation into a tfoX, recA and/or tfoX gene of the cell. In another aspect, the invention includes a heterogenic pool of co-transformed cells comprising two or more co-transformed nucleic acid molecules, wherein the cells are naturally competent and co-transformed with two or more nucleic acid molecules, and wherein at least one of the nucleic acid molecules comprises a selectable marker.

In various embodiments of the above aspects or any other aspect of the invention delineated herein, the naturally competent cells are bacterial cells. In one embodiment, the naturally competent cells are gram negative or gram positive. In one embodiment, the naturally competent cells belong to a phylum selected from the group consisting of Firmicutes, Chroococcales, Bacteriodia, Chlorobi, Deinococci, Actinobacteria, Proteobacteria, and Euryarchaeota. In another embodiment, the naturally competent cells are Bacillus, Cyanobacterium, Lactococcus, Acinetobacter, Neisseria, Haemophilus, Vibrio, or Streptococcus cells. In another embodiment, the naturally competent cells are V. cholerae or S. pneumoniae. In another embodiment, the naturally competent cells are selected from the species listed in Table 1.

In another embodiment, at least one of the nucleic acid molecules comprises at least one arm of homology to a genetic locus of a genome of the naturally competent cells. In some embodiments, the arm of homology has a length of less than about 4 kb. In still another embodiment, at least one of the nucleic acid molecules comprises at least one genome edit. In some embodiments, the genome edit is introduced into a gene involved in natural transformation. In yet another embodiment, the two or more nucleic acid sequences comprise unlinked genetic markers.

In another embodiment, contacting the naturally competent cells with two or more nucleic acid molecules comprises introducing at least one genome edit that optimizes natural transformation. In yet another embodiment, the method of introducing nucleic acid molecules into one or more cells in parallel further comprises repeating steps (a) contacting naturally competent cells with two or more nucleic acid molecules, wherein at least one of the nucleic acid sequences comprises a selectable marker; and (b) selecting for that marker, wherein each repeat comprises a different selectable marker.

In another embodiment, the nucleic acid molecules integrate at a neutral locus. In yet another embodiment, the nucleic acid molecules replace a dispensable gene with an antibiotic resistance marker. In another embodiment, the polynucleotide target is a bacterial artificial chromosome, yeast artificial chromosome, or vector. In still another embodiment, the vector is a mammalian expression vector. In another embodiment, the method of introducing nucleic acid molecules into one or more polynucleotide targets in parallel further comprises transforming a cell. In yet another embodiment, the cell is a bacterial cell, yeast cell, or mammalian cell.

In still another embodiment, the heterogenic pool of co-transformed cells comprises all combinations of the two or more co-transformed nucleic acid sequences.

In another embodiment, at least one selectable marker is a reporter gene or a drug resistance gene. In some embodiments, the drug resistance gene is selected from the group consisting of kanamycin resistance gene, spectinomycin resistance gene, streptomycin resistance gene, chloramphenicol resistance gene, tetracycline resistance gene, and penicillin resistance gene.

In yet another aspect, the invention includes an apparatus for introducing two or more populations of nucleic acid molecules into a population of cells in parallel comprising: a receptacle containing one or more naturally competent cells, wherein the receptacle is configured to produce static conditions that induce natural competence; a container comprising the two or more populations of nucleic acid molecules, wherein the container is fluidically coupled to the receptacle to introduce the two or more populations of nucleic acid molecules into the receptacle for co-transformation into the naturally competent cells; and a container comprising selective growth media to replace the natural competence conditions with selective growth media to select the co-transformed cells.

In one embodiment of the invention, the apparatus further comprises a container comprising a different selective growth media.

### BRIEF DESCRIPTION OF THE DRAWINGS

FIG. 1A is a schematic diagram showing the optimization of co-transformation in Vibrio cholerae at two unlinked genomic locations. The neutral locus targeted for replacement with an Ab^{R} (antibiotic resistance) marker (aka selected product) was VC1807, a transposase pseudogene containing an authentic frameshift.

FIG. 1B is a graph showing co-transformation frequency in assays where the size of homology in the unselected nucleic acid molecule was varied. The unselected nucleic acid molecule—a PCR (polymerase chain reaction) product—contained a transversion point mutation that introduces a premature stop codon into the lacZ gene. Reactions contained the selected product at 30 ng/mL and the unselected product at 3 μg/mL. Data are from at least two biological replicates and are shown as the Mean±Standard Deviation.

FIG. 1C is a graph showing co-transformation frequency in assays where the concentration of the unselected PCR product was varied. Reactions contained PCR products with 3 kb arms of homology and the selected product at 30 ng/mL. Data are from at least two biological replicates and are shown as the Mean±Standard Deviation.

FIG. 1D is a graph showing transformation efficiency when the size of homology in the selected PCR product was varied. Reactions contained the selected product at 30 ng/mL and the unselected product at 3 μg/mL. Data are from at least two biological replicates and are shown as the Mean±Standard Deviation.

FIG. 1E is a graph showing co-transformation frequency when the size of homology in the selected product was varied. Reactions contained the selected product at 30 ng/mL and the unselected product at 3 μg/mL. Data are from at least two biological replicates and are shown as the Mean±Standard Deviation.

FIG. 1F is a graph showing transformation efficiency when the concentration of the selected product was varied. Reactions contained PCR products with 3 kb arms of homology, and the unselected product at 3 μg/mL. Data are from at least two biological replicates and are shown as the Mean±Standard Deviation.

FIG. 1G is a graph showing co-transformation frequency when the concentration of the selected product was varied. Reactions contained PCR products with 3 kb arms of homology, and the unselected product at 3 μg/mL. Data are from at least two biological replicates and are shown as the Mean±Standard Deviation.

FIG. 1H is a graph showing co-transformation frequency in assays using two distinct unselected genetic markers, where one was in lacZ, which was ˜500 kb from the selected marker on the genome, and the other was upstream of VCA0063, which was on a distinct chromosome from the selected marker. Reactions contained PCR products with 3 kb arms of homology, the selected product at 30 ng/mL and the unselected product at 3 μg/mL. Data are from at least two biological replicates and are shown as the Mean±Standard Deviation.

FIG. 1I is a graph showing co-transformation frequency in assays using unselected products to generate deletions of the indicated size in the lacZ gene. Reactions contained PCR products with 3 kb arms of homology, the selected product at 30 ng/mL and the unselected product at 3 μg/mL. Data are from at least two biological replicates and are shown as the Mean±Standard Deviation.

FIG. 1J is a graph showing co-transformation frequency of insertion mutations as measured by reverting strains with deletions in lacZ of the indicated size back to WT (wildtype). Reactions contained PCR products with 3 kb arms of homology, the selected product at 30 ng/mL and the unselected product at 3 μg/mL. Data are from at least two biological replicates and are shown as the Mean±Standard Deviation.

FIG. 2A is a schematic diagram showing the approach described herein to randomize six (N6) or 30 (N30) base pairs in the lacZ gene by co-transformation and deep-sequencing of the N6 or N30 regions.

FIG. 2B is a graph showing frequency of number of randomized bases in the lacZ gene following two cycles (C1 and C2) of co-transformation with the N6 and N30 PCR products.

FIG. 2C is a graph showing the composition of the N6 regions in the input PCR product and output co-transformant pools as measured by divergence of sequences from the WT consensus sequence.

FIG. 2D is a graph showing the composition of the N30 regions in the input PCR product and output co-transformant pools as measured by divergence of sequences from the WT consensus sequence.

FIG. 2E is a graph showing linear regression of the abundance of all 4096 N6 mers, excluding the WT sequence, in the input PCR product and output co-transformant pool for the N6 C1 sample.

FIG. 3A is a schematic diagram showing the strategy for generating complex heterogenic mutant populations using co-transformation and the five genetic loci targeted in an experiment described herein.

FIG. 3B is a schematic diagram depicting the roles of targeted loci in V. cholerae natural transformation. TfoX and HapR are regulators that control the indicated processes.

FIG. 3C is a graph showing the distribution of genome edits in the population following two cycles of co-transformation (C1 and C2), and two rounds of selection with just an Ab^{R} conferring selected marker (R1 and R2). Co-transformation was used to introduce genome edits into a population of cells in multiplex. PCR products for each mutation were mixed at equimolar concentrations with a selectable marker in transformation reactions. Multiple cycles of MuGENT were carried out by using selected products to alter the antibiotic resistance cassette at the neutral locus at each cycle. Transformants were screened by multiplex allele-specific colony (MASC) PCR, and after a single cycle of co-selection (C1), ˜50% of the population was found to have at least one genetic edit. After a second cycle of co-selection (C2/R0), ˜90% of the population contained at least one edit and ˜4% had edits at all five loci.

FIG. 3D is a graph showing the frequency of each genome edit following selection.

FIG. 3E is a panel of graphs showing the final biomass on chitin and transformation efficiencies from transformation assays. The grid under the X-axis indicates the genotype of strains. A filled box indicates the presence of a genome edit and the color indicates the strength of the edited RBS (ribosome binding site). Black is used for mutS, as this gene was targeted for inactivation. Data are from four independent biological replicates and are shown as the Mean±Standard Deviation.

FIG. 4A is a graph showing frequencies of genome edits in the four pht genes in WT Streptococcus pneumoniae after co-transformation with an antibiotic resistance marker for 1, 3 and 5 rounds of co-transformation. The four pht genes were targeted by using PCR products that introduce tandem stop codons into each locus.

FIG. 4B is a graph showing frequencies of genome edits in the four pht genes in an MMR (mismatch repair) deficient S. pneumoniae strain.

FIG. 4C is an electrophoretic gel showing MASC (multiplex allele-specific colony) PCR of all 16 possible pht mutant strains made in the wildtype background. A band indicates the presence of a genome edit.

FIG. 5A is a schematic diagram for RBS optimization at tfoX, recA, hapR and dprA showing the bases that were randomized. The first RBS shown for each gene represents the WT RBS. RBS strengths shown are from the ribosome binding site calculator and based on an arbitrary scale of 0-100,000.

FIG. 5B is schematic of the nucleic sequence design of transversion (TAA) and transition (TAG) mutations in lacZ, which result in premature stop codons. Transition mutations are more efficiently repaired by MMR compared to transversion mutations.

FIG. 5C is a graph showing co-transformation frequency for these mutations in WT and the MMR deficient mutS deletion strain, demonstrating little to no effect of MMR on co-transformation of V. cholerae. Data are from two biological replicates and are shown as the Mean±Standard Deviation.

FIG. 6A is a schematic diagram showing co-transformation and recombination of a bacterial genome with selected and unselected markers generated from PCR products.

FIG. 6B is a schematic diagram showing the recombination of a bacterial genome with an unselected marker from a PCR product and co-transformation of a plasmid carrying the selectable marker for kanamycin resistance.

FIG. 6C is a graph showing the co-transformation frequency where the selected marker is a PCR product in which VC1807 is replaced with a kanamycin resistance gene (left) and plasmid pBAD18 containing a kanamycin resistance gene (right). The unselected marker is shown in FIGS. 6A and 6B.

FIG. 6D is a graph showing the transformation efficiency of TG1 (recA+) cells and DH5α (recA−) cells.

FIG. 7 is a graph showing co-transformation frequency in co-transformation mutagenesis of a bacterial artificial chromosome in a V. cholerae host strain.

### DEFINITIONS

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

By “arm of homology” is meant a portion of a nucleic acid sequence that is homologous to another nucleic acid sequence. In one embodiment, a nucleic acid sequence comprises at least one arm of homology to a portion of a genome of the naturally competent cells.

By “co-transformation” is meant introduction of two or more nucleic acid sequences into a cell.

By “genome edit” is meant an alteration to a genomic locus. The alteration can include one or more of an addition, deletion, substitution and rearrangement. In one embodiment, the genome edit is introduced through co-transformation.

By “genomic locus” or “genomic loci” is meant one or more locations, positions or sequences in a genome, respectively. In one embodiment, the location, position or sequence of the genomic locus is in a gene or a regulatory region of the gene.

By “genetic linkage” or “linked genetic markers” is meant two or more genetic loci that are located proximal to one another on the chromosome or in the genome. Decreased frequency of cross-over between linked genes indicates a smaller distance separating the genetic loci.

By “unlinked genetic markers” is meant two or more genetic loci that have a recombination frequency independent of distance separating the genetic loci.

By “genetic locus” or “genetic loci” is meant one or more locations, positions or sequences in a gene, respectively.

As used herein, “phenotype” refers to the entire physical, biochemical, and physiological makeup of a cell, e.g., having any one trait or any group of traits.

By “homologous recombination” is meant a type of genetic recombination in which nucleic acid sequences are exchanged between two similar or identical molecules of DNA.

By “naturally competent cell” is meant a cell that is capable of taking up extracellular nucleic acid sequences without mechanical permeabilization of the cell membrane. Competence may be induced in the cell by high cell density culturing and/or nutritional limitation, and conditions associated with the stationary phase of bacterial growth.

By “optimizing natural transformation” is meant increasing the natural transformative abilities or potential of a cell already capable of natural transformation to undergo transformation more readily or with greater efficiency. Examples of such optimization include increasing expression of genes that promote natural transformative abilities or potential, and/or decreasing expression of genes that inhibit or block natural transformative abilities or potential.

By “selectable agent” is meant an agent that produces a selection pressure on cells exposed to the agent. For example, the selective agent is an antibiotic agent, such as kanamycin, spectinomycin, streptomycin, ampicillin, chloramphenicol, tetracycline, and penicillin, and exposure of cells that are transformed with an antibiotic resistance gene are resistant to the antibiotic agent.

By “selectable marker” is meant a gene that confers a phenotype or trait to the cells harboring the selectable marker. A selectable marker can include, but is not limited to, a reporter gene (e.g., lacZ), and a drug resistance gene (antibiotic resistance gene).

By “selective growth media” is meant a growth media comprising one or more selectable agents.

By “static conditions” is meant an incubation or culture environment where growth of the cells is minimal and activities related to growth are decreased.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “base substitution” is meant a substituent of a nucleobase polymer that does not cause significant disruption of the hybridization between complementary nucleotide strands.

By “fragment” is meant a portion of a polynucleotide or nucleic acid molecule. This portion contains, preferably, at least 10%, 20%, 30%, 40%, 50%, 60%, 70%, 80%, or 90% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. A “fragment” of a nucleic acid molecule may be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

By “identity” is meant the nucleic acid sequence identity between a sequence of interest and a reference sequence. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. That is, a nucleic acid is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel.

The term “nucleic acid” refers to deoxyribonucleic acids (DNA) or ribonucleic acids (RNA) thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

By “reference” is meant a standard or control condition.

A “reference sequence” is a defined sequence used as a basis for sequence comparison.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

The invention generally features methods for transforming a naturally competent micro-organism with two or more nucleic acid molecules and cells comprising these molecules.

The present invention is based, in part, on the discovery that naturally competent cells are transformable with multiple nucleic acid sequences.

Editing bacterial genomes is an essential tool in research and synthetic biology applications. Here, Multiplex Genome Editing by Natural Transformation (MuGENT), a method for accelerated evolution based on the co-transformation of unlinked genetic markers in naturally competent microorganisms, is described. It was found that natural co-transformation of a selected and unselected nucleic acid molecules allowed for scarless genome editing via recombination of the unselected nucleic acid molecule at unprecedented frequencies of ˜50%. Using nucleic acid molecules with randomized nucleotides, no evidence for bias during natural co-transformation was found, indicating that this method can be used for directed evolution studies. Furthermore, it was found that natural co-transformation was an effective method for multiplex genome editing. Since MuGENT does not require selection at edited loci in cis, output mutant pools are highly complex, where strains have any number and combination of the multiplexed genome edits. We demonstrate the utility of this technique in metabolic and phenotypic engineering by optimizing natural transformation in V. cholerae. This was accomplished by combinatorially editing the genome via gene deletions, promoter replacements and by tuning translation initiation of five genes involved in the process of natural competence and transformation. MuGENT allowed for generation of a complex mutant pool in one week, and resulted in the selection of a genetically edited strain with a 30-fold improvement in natural transformation. We also demonstrate the efficacy of this technique in S. pneumoniae and highlight the potential for MuGENT to be used in multiplex genetic interaction analysis. Thus, MuGENT is a broadly applicable platform for accelerated evolution and genetic interaction studies in diverse naturally competent species.

### MuGENT

The ability to generate mutants is essential in microbiology research. Although methods have been developed for making defined single mutations in bacterial genomes, methods for simultaneously generating multiple defined mutations, i.e., multiplex genome editing, have been limited to model species like E. coli. Diverse microbial species have the ability to naturally take up exogenous DNA and integrate it into their genome—a process known as natural transformation. While natural transformation has been exploited for making single mutations, it has not previously been used for multiplex genome editing.

Directed evolution through genome editing is an increasingly important method used in pharmaceutical and industrial research to improve the ability of microbes to produce biomolecules or to degrade wastes. This is typically done through the optimization of expression of genes within relevant biochemical pathways. Current technologies for editing microbial genomes are laborious and limited to the sequential editing of single loci, therefore development of technologies that allow for simultaneous editing of multiple loci would be of great value to our society. While technologies have been developed for multiplexed genome editing in a handful of model bacteria like E. coli, these technologies are not amenable to microbes of industrial importance. A powerful technology is described herein that allows for the simultaneously editing of multiple loci in naturally transformable microbes, called Multiplexed Genome Editing via Natural Transformation (MuGENT).

Natural transformation is the ability to take up and integrate exogenously added DNA and is a trait shared by most industrially important microbes. MuGENT is based on the co-transformation of a selectable marker and a set of unmarked, genetically altered loci designed to improve a phenotype of interest. For example, the expression level of each gene within a biosynthetic pathway can be simultaneously varied, regardless of their location within the genome, in order to optimize end-product production. In a proof-of-principle experiment, five unlinked loci were simultaneously edited. Because each genetic alteration occurs independently during the cotransformation, a single experiment yields a pool of mutants comprising all possible combinations of the mutations. This makes MuGENT an exceptionally powerful platform for directed evolution of microbes. For complex phenotypes involving dozens of genes, iterative cycles of MuGENT can be done. This allows for the testing of a mutational space that is much larger than what can be tested in a single experiment. Thus, MuGENT holds great promise for the accelerated, directed evolution of microbes on extraordinarily short timescales.

### Natural Competence

Natural competence and transformation is a trait shared by diverse microbial species. It involves the uptake of DNA from the extracellular environment followed by integration of this DNA into the genome by homologous recombination. During natural transformation, only a fraction of cells in the population become competent and are transformed. It has previously been demonstrated that it is possible to co-transform unlinked markers in naturally competent bacteria, indicating that each competent cell has the ability to take up multiple DNA molecules. The use of co-transformation for multiplex genome editing applications, however, has not previously been explored. Here, natural co-transformation was optimized and demonstrated its use as a method for multiplex genome editing in naturally competent V. cholerae and S. pneumoniae.

The invention generally provides a method for introducing multiple nucleic acid sequences into one or more naturally competent cells in parallel. In one aspect, the invention includes a method of introducing nucleic acid sequences into one or more cells in parallel comprising the steps of: i) obtaining naturally competent cells; ii) contacting the naturally competent cells with two or more nucleic acid sequences, wherein at least one of the nucleic acid sequences comprises a selectable marker; and iii) incubating the cells with growth medium selective for the selectable marker, wherein two or more nucleic acid sequences are introduced into the cells in parallel. In one embodiment, the method further comprises repeating steps ii) and iii), wherein each repeat comprises a different selectable marker. In one aspect, the invention includes a method of introducing nucleic acid sequences into one or more cells in parallel comprising the steps of: i) obtaining naturally competent cells; ii) adding two or more nucleic acid sequences to naturally competent cells, wherein at least one of the nucleic acid sequences comprises a selectable marker; and iii) incubating the cells with growth medium selective for the selectable marker, wherein two or more nucleic acid sequences are introduced into the cells in parallel. In another aspect, the invention includes contacting naturally competent cells with two or more nucleic acid sequences, wherein at least one of the nucleic acid sequences comprises a selectable marker, to create a heterogenic pool of co-transformed cells comprising two or more co-transformed nucleic acid sequences.

While many cells are naturally competent, cells may be further conditioned to accept multiple nucleic acid sequences. The cells may be bacterial cells, yeast cells, or mammalian cells. In another embodiment, obtaining naturally competent cells comprises incubating cells under static conditions. The static conditions can include those that minimize growth and activities of the cells.

In some embodiments, the naturally competent cells are selected from the group consisting of Firmicutes, Chroococcales, Bacteriodia, Chlorobi, Deinococci, Actinobacteria, Proteobacteria, and Euryarchaeota. In some other embodiments, the naturally competent cells are selected from the species listed in Table 1.

| | | | | | | DprA | | ComEC | |
|---|---|---|---|---|---|---|---|---|---|
| Spec. | | | | Shot | | Swiss | DprA | Swiss | ComEC |
| tree | | | Chromo. | gun | | Prot | Locus | Prot | Locus |
| order | Phylum | Species name | Accession | seq. | RefSeq | ID | Tag | ID | Tag |
| 1 | Firmicutes | Staphylococcus | BA000017.4 | | NC_002758 | Q99UM2 | SAV1249 | Q99TR1 | SAV1588 |
| | (other) | aureus Mu50 | | | | | | | |
| 2 | Firmicutes | Bacillus | AE017333.1 | | NC_006322 | Q65JN8 | BLi01031 | Q65H43 | BLi02750 |
| | (Bacilli) | licheniformis | | | | | | | |
| | | DSM 13 | | | | | | | |
| 3 | | Bacillus subtilis | AL009126.3 | | NC_000964 | P39813 | BSU16110 | P39695 | BSU25570 |
| | | 168 | | | | | | | |
| 4 | | Bacillus | CP000560.1 | | NC_009725 | A7Z4N1 | RBAM_015940 | A7Z6X1 | RBAM_023870 |
| | | amyloliquefaciens | | | | | | | |
| | | FZB42 | | | | | | | |
| 5 | Firmicutes | Lactobacillus | CR936503.1 | | NC_007576 | Q38WY7 | LCA_0992 | Q38WR1 | LCA_1069 |
| | (other) | sakei 23K | | | | | | | |
| 6 | | Leuconostoc | CP003851.1 | | NC_018673 | K0D7D3 | C270_04415 | K0D947 | C270_01930 |
| | | camosum JB16 | | | | | | | |
| 7 | Firmicutes | Streptococcus | AE014133.2 | | NC_004350 | Q8DUD4 | SMU_1001 | Q8DV79 | SMU_626 |
| | (Streptococci) | mutans UA159 | | | | | | | |
| 8 | | Streptococcus | CP000419.1 | | NC_008532 | Q03KX5 | STER_0922 | Q03JE9 | STER_1520 |
| | | thermophilus | | | | | | | |
| 9 | | Streptococcus | FR873482.1 | | | F8LQ76 | SALIVA_1183 | F8LRF3 | SALIVA_1617 |
| | | salivarius | | | | | | | |
| | | JIM8777 | | | | | | | |
| 10 | | Streptococcus | CP003295.1 | | NC_016826 | H6P9S9 | Sinf_0910 | H6P9F4 | Sinf_0543 |
| | | infantarius CJ18 | | | | | | | |
| 11 | | Streptococcus | HE613569.1 | | NC_016749 | H2A6E8 | SMA_1021 | H2A5D3 | SMA_0621 |
| | | macedonicus | | | | | | | |
| | | ACA-DC 198 | | | | | | | |
| 12 | | Streptococcus | | AEEL | | | | | |
| | | bovis ATCC | | | | | | | |
| | | 700338 | | | | | | | |
| 13 | | Streptococcus | FR720602.1 | | NC_015291 | F2QD07 | SOR_0865 | F2QE02 | SOR_1213 |
| | | oralis Uo5 | | | | | | | |
| 14 | | Streptococcus | AE007317.1 | | NC_003098 | Q8DPI7 | spr1144 | Q8DQ40 | spr0857 |
| | | pneumoniae R6 | | | | | | | |
| 15 | | Streptococcus | FN568063.1 | | NC_013853 | D3H9C3 | smi_1223 | D3H8K8 | smi_0956 |
| | | mitis B6 | | | | | | | |
| 16 | | Streptococcus | AP010969.1 | | NC_018073 | I4E040 | SCIM_0876 | I4E076 | SCIM_0912 |
| | | intermedius | | | | | | | |
| | | JTH08 | | | | | | | |
| 17 | | Streptococcus | | ALJO | | | | | |
| | | anginosus | | | | | | | |
| | | SK1138 | | | | | | | |
| 18 | | Streptococcus | | AEVC | | | | | |
| | | cristatus | | | | | | | |
| 19 | | Streptococcus | CP000387.1 | | NC_009009 | A3CN35 | SSA_1185 | A3CLU7 | SSA_0716 |
| | | sanguinis SK36 | | | | | | | |
| 20 | | Streptococcus | CP000725.1 | | NC_009785 | A8AXH5 | SGO_1198 | A8AYL9 | SGO_1601 |
| | | gordonii Challis | | | | | | | |
| 21 | Chroococcales | Thermosyne- | BA000039.2 | | NC_004113 | Q8DJ00 | tlr1431 | Q8DI88 | tll1702 |
| | (Cyanobac- | chococcus | | | | | | | |
| | teria) | elongatus BP-1 | | | | | | | |
| 22 | | Synechocystis | BA000022.2 | | NC_000911 | P73345 | slr1197 | P73100 | sll1929 |
| | | spp. PCC6803 | | | | | | | |
| 23 | | Synechococcus | AP008231.1 | | NC_006576 | Q5N3V3 | syc0827_c | Q5N1I4 | syc1646_c |
| | | elongatus PCC | | | | | | | |
| | | 6301 | | | | | | | |
| 24 | Bacteriodia | Porphyromonas | CP000510.1 | | NC_008709 | Q7MXB1 | PG_0295 | Q7MUD5 | PG_1594 |
| | | gingivalis W83 | | | | | | | |
| 25 | | Chlorobium | CP001097.1 | | NC_010803 | B3EFC5 | Clim_0314 | B3EDH5 | Clim_1551 |
| | | limicola DSM | | | | | | | |
| | | 245 | | | | | | | |
| 26 | Chlorobi | Chlorobium | AE006470.1; | | NC_002932 | Q8KFR7 | CT0255 | Q8KCP6 | CT1367 |
| | | tepidum TLS | AL646053.1 | | | | | | |
| 27 | | Deinococcus | AE000513; | | | Q9RY31 | DR_0120 | Q9RTB1 | DR_1854 |
| | | radiodurans R1 | AE001825 | | | | | | |
| 28 | Deinococci | Thermus | AE017221.1 | | NC_005835 | Q72GH5 | TT_C1873 | Q72H92 | TT_C1603 |
| | | thermophilus | | | | | | | |
| | | HB27 | | | | | | | |
| 29a | Actinobacteria | Streptomyces | CP002993.1 | | NC_015953 | G2NJ75 | SACTE_4823 | G2NE25 | SACTE_1980 |
| | | virginiae^{b} (spp.) | | | | | | | |
| | | SirexAA-E | | | | | | | |
| 29b | | Streptomyces | | | | | | | |
| | | kasugaensis | | | | | | | |
| 30 | Proteobacteria | Thiobacillus | | ARDU | | | | | |
| | (b) | thioparus DSM | | | | | | | |
| | | 505 | | | | | | | |
| 31 | | Ralstonia | AL646052.1 | | NC_003295 | Q8Y3B2 | RSc0068 | Q8Y0C4 | RSc1120 |
| | | solanacearum | | | | | | | |
| | | GMI1000 | | | | | | | |
| 32 | | Achromobacter | | AGUF | | | | | |
| | | spp. SY8 | | | | | | | |
| 33 | | Neisseria | AE002098.2 | | NC_003112 | Q9K1K1 | NMB0116 | Q9K0B2 | NMB0702 |
| | | meningitidis | | | | | | | |
| | | MC58 | | | | | | | |
| 34 | | Neisseria | AE004969.1 | | NC_002946 | Q5F5Q2 | NGO1865 | Q5F9W1 | NGO0276 |
| | | gonorrhoeae FA | | | | | | | |
| | | 1090 | | | | | | | |
| 35 | | Kingella kingae | | AFHS | | | | | |
| | | ATCC 23330 | | | | | | | |
| 36 | | Kingella | | AEWV | | | | | |
| | | denitrificans | | | | | | | |
| | | ATCC 3339 | | | | | | | |
| 37 | Proteobacteria | Xylella | CP000941.1 | | NC_010513 | B0U4M6 | Xfasm12_1931 | B0U4R0 | Xfasm12_0386 |
| | (g) | fastidiosa M12 | | | | | | | |
| 38 | | Legionella | AE017354.1 | | NC_002942 | Q5ZSC2 | Ipg2597 | Q5ZXV5 | Ipg0626 |
| | | pneumophila | | | | | | | |
| | | Philadelphia 1 | | | | | | | |
| 39 | | Acinetobacter | | AMIC | | | | | |
| | | baylyi TG19579 | | | | | | | |
| 40 | | Pseudomonas | CP000094.2 | | NC_007492 | Q3KKE3 | Pfl01_0019 | Q3K8I9 | Pfl01_4178 |
| | | fluorescens Pf0-1 | | | | | | | |
| 41 | | Pseudomonas | CP000304.1 | | NC_009434 | A4VFI0 | PST_0023 | A4VMT8 | PST_2639 |
| | | stutzeri A1501 | | | | | | | |
| 42 | | Azotobacter | CP001157.1 | | NC_012560 | C1DFW0 | Avin_00190 | C1DR16 | Avin_14730 |
| | | vinelandii DJ; | | | | | | | |
| | | ATCC BAA- | | | | | | | |
| | | 1303 | | | | | | | |
| 43 | | Pseudomonas | CP000680.1; | | NC_009439 | A4XNB5 | Pmen_0057 | A4XSQ9 | Pmen_1611 |
| | | mendocina ymp | AE003853.1 | | | | | | |
| 44 | | Vibrio fischeri | CP000020.2; | | NC_006840; | Q5E1R0 | VF_2541 | Q5E0F3 | VF_A0423 |
| | | ES114 | CP000021.2 | | NC_006841 | | | | |
| 45 | | Vibrio cholerae | AE003852.1 | | NC_002505; | Q9KVU1 | VC_0048 | Q9KQW8 | VC_1879 |
| | | N16961 | | | NC_002506 | | | | |
| 46 | | Vibrio vulnificus | AE016795.3; | | NC_004459; | Q8DDE1 | VV1_1050 | Q8DAV3 | VV1_2084 |
| | | CMCP6 | AE016796.2 | | NC_004460 | | | | |
| 47 | | Vibrio | BA000032.2; | | NC_004605; | Q87KD7 | | Q87R17 | |
| | | paraheamolyticus | BA000031.2 | | NC_004603 | | | | |
| | | O3:K6 | | | | | | | |
| 48 | | Vibrio spp. | CP001805.1; | | NC_013456; | A7K4Z8 | VEA_002059 | A7K283 | VEA_003988 |
| | | EX25 | CP001806.1 | | NC_013457 | | | | |
| 49 | | Escherichia | U00096.2 | | NC_000913 | P30852 | b4473 | P37443 | b0913 |
| | | coli K-12 | | | | | | | |
| 50 | | Gallibacterium | CP002667.1 | | NC_015460 | F4H9U4 | UMN179_01231 | F4HDN9 | UMN179_00665 |
| | | anatis UMN179 | | | | | | | |
| 51 | | Actinobacillus | CP003875.1 | | NC_018690 | K0G6Q5 | ASU2_09565 | K0G559 | ASU2_03145 |
| | | suis H91-0380 | | | | | | | |
| 52 | | Actinobacillus | CP000569.1 | | NC_009053 | A3N310 | APL_1712 | A3N0C8 | APL_0766 |
| | | pleuropneumoniae | | | | | | | |
| | | L20 | | | | | | | |
| 53 | | Haemophilus | CP001321.1 | | NC_011852 | B8F725 | HAPS_1573 | B8F483 | HAPS_0467 |
| | | parasuis | | | | | | | |
| | | SH0165 | | | | | | | |
| 54 | | Haemophilus | L42023.1 | | NC_000907 | P43862 | HI_0985 | P44408 | HI_0061 |
| | | influenzae Rd | | | | | | | |
| | | KW20 | | | | | | | |
| 55 | | Haemophilus | FQ312002.1 | | NC_015964 | E1W3Z1 | PARA_09770 | E1W502 | PARA_13430 |
| | | parainfluenzae | | | | | | | |
| | | T3T1 | | | | | | | |
| 56 | | Aggregatibacter | CP001607.1 | | NC_012913 | C6AQ11 | NT05HA_1586 | C6ANC3 | NT05HA_0957 |
| | | aphrophilus | | | | | | | |
| | | NJ8700 | | | | | | | |
| 57 | | Aggregatibacter | CP001733.1 | | NC_013416 | C9R1W7 | D11S_0393 | C9R469 | D11S_1223 |
| | | actinomycetemc | | | | | | | |
| | | omitans D11S-1 | | | | | | | |
| d | Firmicutes | Bacillus | | | | | | | |
| | (Bacilli) | stearothermophilus | | | | | | | |
| d | Firmicutes | Lactobacillus | | | | | | | |
| | (other) | lactis | | | | | | | |
| d | | Thermoactinomyces | | | | | | | |
| | | vulgaris | | | | | | | |
| e | Firmicutes | Streptococcus | | AEVC | | | | | |
| | (Streptococci) | constellatus | | | | | | | |
| | | ATCC 51100 | | | | | | | |
| e | | Streptococcus | | | | | | | |
| | | infantis | | | | | | | |
| d | Chroococcales | Nostoc | | | | | | | |
| | | muscorum | | | | | | | |
| e | Deinococci | Thermus | | | | | | | |
| | | aquaticus | | | | | | | |
| d | | Thermus | | | | | | | |
| | | caldophilus | | | | | | | |
| d | | Thermus flavus | | | | | | | |
| e | Proteobacteria | Eikenella | | | | | | | |
| | (b) | corrodens | | | | | | | |
| d | | Thiobacillus | | | | | | | |
| | | spp. Strain Y | | | | | | | |
| e | Proteobacteria | Cardiobacterium | | | | | | | |
| | (g) | hominis | | | | | | | |
| d | | Moraxella spp. | | | | | | | |
| d | | Pseudomonas | | | | | | | |
| | | alcaligenes | | | | | | | |
| d | | Pseudomonas | | | | | | | |
| | | pseudoalcaligenes | | | | | | | |
| e | | Pseudomonas | | | | | | | |
| | | spp. | | | | | | | |
| f | Proteobacteria | Campylobacter | | | | | | | |
| | (e) | coli | | | | | | | |
| f | | Campylobacter | | | | | | | |
| | | jejuni | | | | | | | |
| f | | Helicobacter | AE000511.5 | | NC_000915.1 | | | | |
| | | pylori | | | | | | | |
| g | Proteobacteria | Agrobacterium | CP000628.1 | | NC_011985.1 | | | | |
| | (a) | tumefaciens | | | | | | | |
| d | | Methylobacterium | | | | | | | |
| | | organophilum | | | | | | | |
| g | | Bradyrhizobium | AP012206.1 | | NC_017249.1 | | | | |
| | | japonicum | | | | | | | |
| h | Euryarchaeota | Methanobacterium | | | | | | | |
| | | thermoauto- | | | | | | | |
| | | trophicum | | | | | | | |
| h | | Methanococcus | CP002057.1 | | NC_01422.1 | | | | |
| | | voltae | | | | | | | |

Genome editing of multiple genes is an essential tool in research and synthetic biology applications. It is important for producing strains of cells with desired phenotypes or traits or expression of particular recombinant products. Accelerated evolution based on co-transformation of unlinked genetic markers in naturally competent microorganisms is one approach for multiplex genome editing. In one embodiment, two or more of the nucleic acid sequences comprise unlinked genetic markers.

In one embodiment, the naturally competent cells are contacted with at least one of the nucleic acid sequences that comprise at least one arm of homology to a genetic locus of a genome of the naturally competent cells. The arm of homology can have a length of less than about 5 kb, 4.5 kb, 4 kb, 3.5 kb, 3 kb, 2.5 kb, 2 kb, 1.5 kb, 1 kb, 900 bases, 800 bases, 700 bases, 600 bases, 500 bases, or less. In an exemplary embodiment, the arm of homology has a length of less than about 4 kb. The arm of homology can have a length in the range of about 1 kb to about 4 kb, and about 1.5 kb to about 3 kb.

The invention also includes at least one of the nucleic acid sequences comprising at least one genome edit. In certain embodiments, the genome edit is introduced into a gene involved in natural transformation. The introduction of a genome edit can alter the activity of the gene, such as increased expression to promote natural transformation. In another embodiment, contacting the naturally competent cells with two or more nucleic acid sequences comprises introducing at least one genome edit that optimizes natural transformation.

The selectable marker is a gene that confers a phenotype or trait to the cells harboring the selectable marker. A selectable marker can include, but is not limited to, a reporter gene (e.g., lacZ), and a drug resistance gene (antibiotic resistance gene). In one embodiment, the drug resistance gene is selected from the group consisting of kanamycin resistance gene, spectinomycin resistance gene, streptomycin resistance gene, chloramphenicol resistance gene, tetracycline resistance gene, and penicillin resistance gene.

Co-Transformed Cells

Also included in the invention is a composition of the naturally competent cells after introduction of the nucleic acid sequences. In one aspect, the invention includes a heterogenic pool of co-transformed cells comprising two or more co-transformed nucleic acid sequences, wherein the cells are naturally competent and co-transformed with two or more nucleic acid sequences, and wherein at least one of the nucleic acid sequences comprises a selectable marker.

In one embodiment, at least one selectable marker is a reporter gene or a drug resistance gene. When the selectable marker is a drug resistance gene, the drug resistance gene is selected from the group consisting of kanamycin resistance gene, spectinomycin resistance gene, streptomycin resistance gene, chloramphenicol resistance gene, tetracycline resistance gene, and penicillin resistance gene.

The heterogenic pool of co-transformed cells includes naturally competent cells selected from the group consisting of Firmicutes, Chroococcales, Bacteriodia, Chlorobi, Deinococci, Actinobacteria, Proteobacteria, and Euryarchaeota. In some other embodiments, heterogenic pool of co-transformed cells includes naturally competent cells selected from the species listed in Table 1.

The nucleic acid sequences used to produce the co-transformed naturally competent cells can include two or more nucleic acid sequences comprising unlinked or linked genetic markers. In some embodiments, at least one of the nucleic acid sequences comprises at least one arm of homology to a genetic locus of a genome of the naturally competent cells. In these instances, the the arm of homology can have a length of less than about 5 kb, 4.5 kb, 4 kb, 3.5 kb, 3 kb, 2.5 kb, 2 kb, 1.5 kb, 1 kb, 900 bases, 800 bases, 700 bases, 600 bases, 500 bases, or less. In an exemplary embodiment, the arm of homology has a length of less than about 4 kb. The arm of homology can have a length in the range of about 1 kb to about 4 kb, and about 1.5 kb to about 3 kb.

The heterogenic pool of co-transformed naturally competent cells can include at least one of the nucleic acid sequences comprises at least one genome edit. The genome edit can further be introduced into a gene involved in natural transformation. When this occurs, the heterogenic pool of co-transformed naturally competent cells are optimized for natural transformation.

In another embodiment, the heterogenic pool comprises all possible combinations of the two or more nucleic acid sequences. Thus, the co-transformed cells represent all the recombination possibilities with the two or more nucleic acid sequences.

### Apparatus

In another aspect, the invention includes an apparatus for introducing two or more populations of nucleic acid sequences into a population of cells in parallel comprising: a receptacle containing one or more naturally competent cells, wherein the receptacle is configured to produce static conditions that induce natural competence; a container comprising the two or more populations of nucleic acid sequences, wherein the container is fluidically coupled to the receptacle to introduce the two or more populations of nucleic acid sequences into the receptacle for co-transformation into the naturally competent cells; and a container comprising selective growth media to replace the natural competence conditions with selective growth media to select the co-transformed cells. In one embodiment, the apparatus further comprises a container comprising a different selective growth media.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Optimization of Natural Co-Transformation

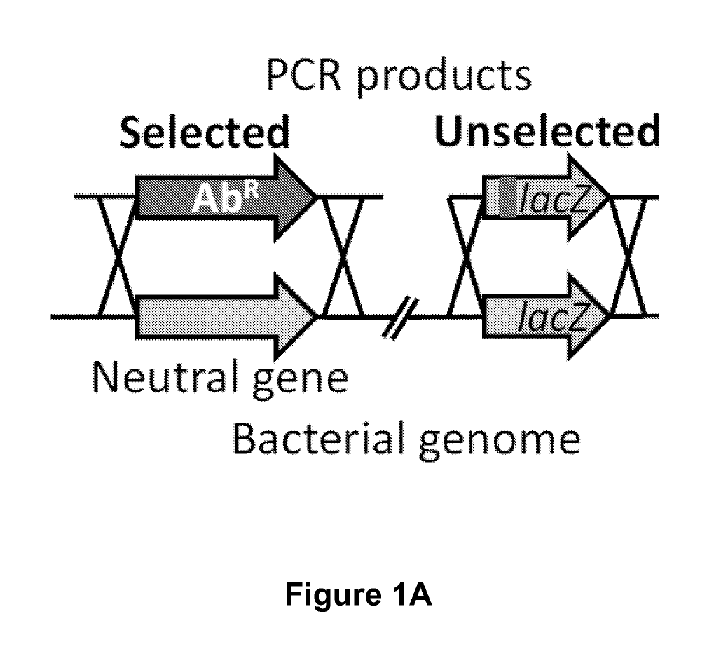

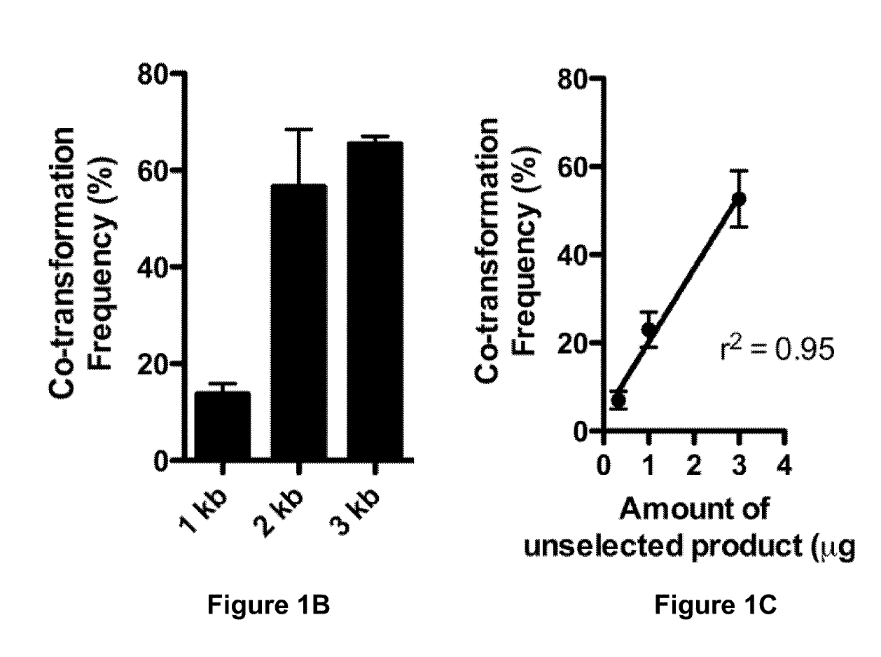

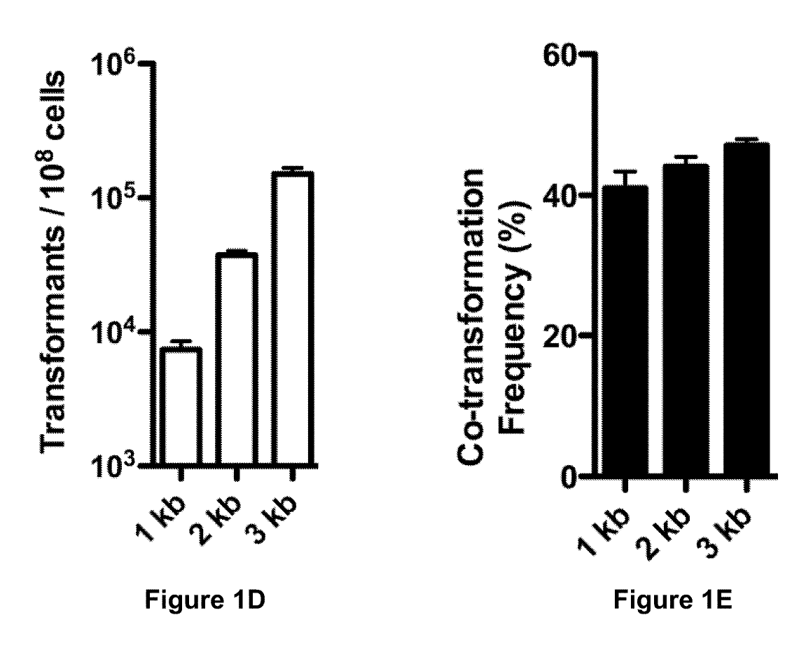

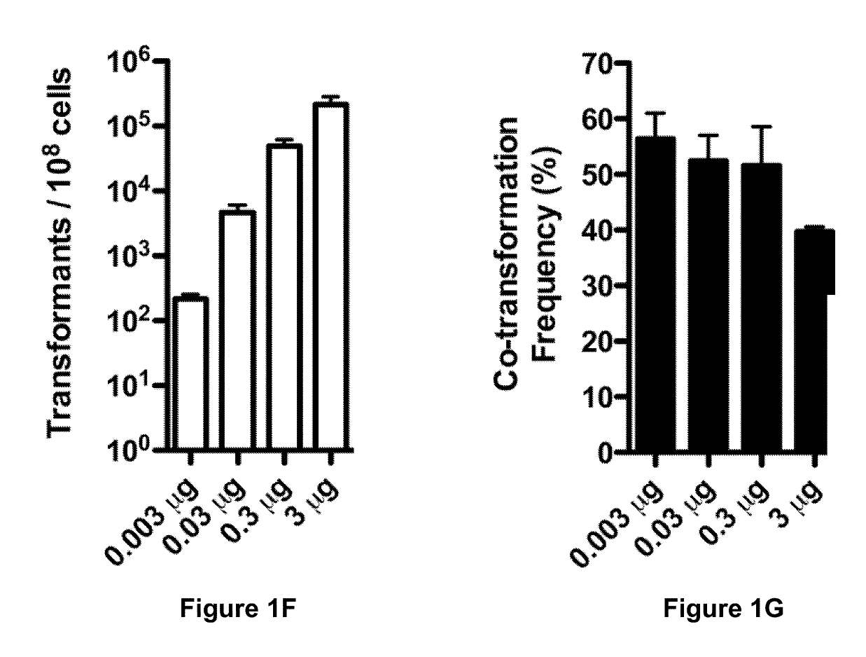

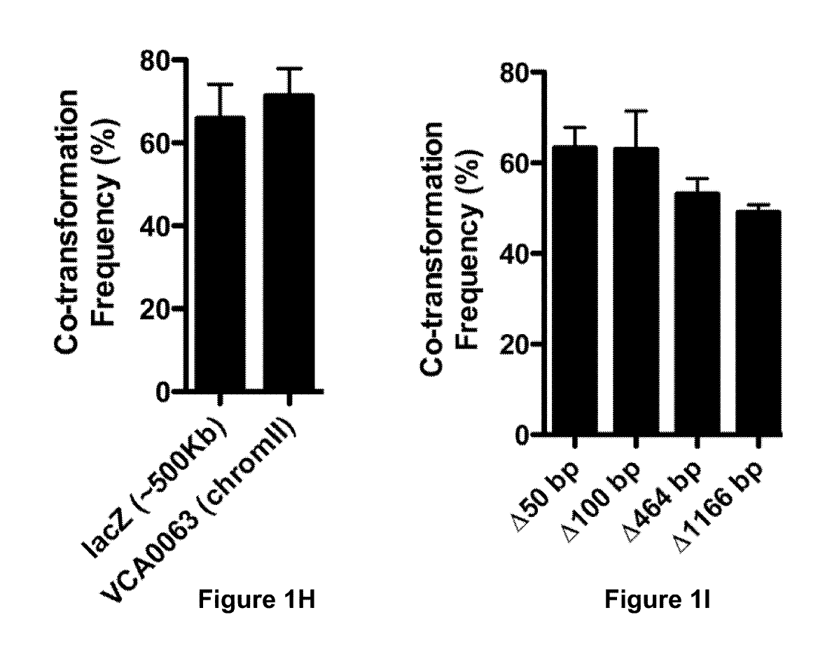

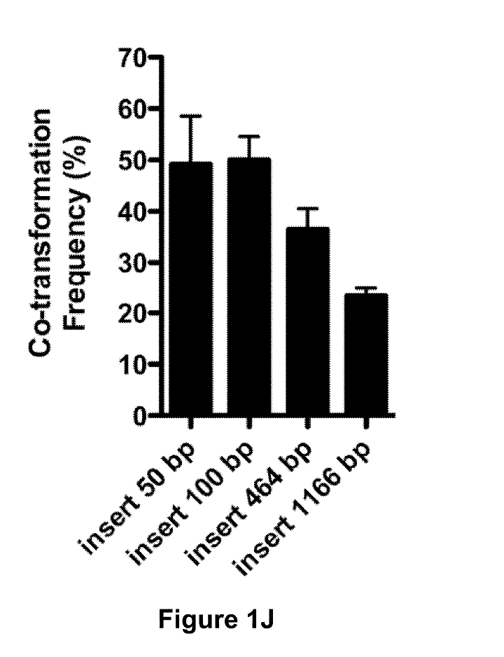

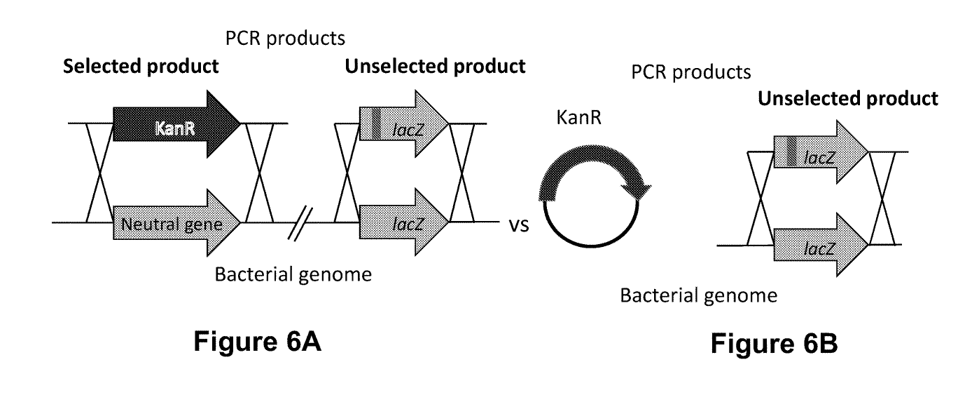

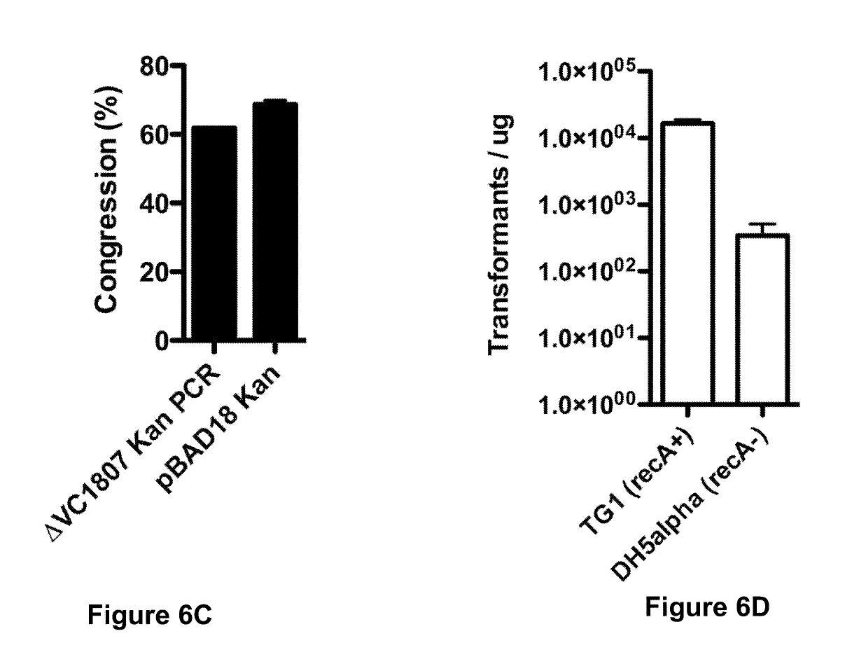

As a first step, the co-transformation of two unlinked markers in V. cholerae was optimized, where one marker was selected and screened for integration of the other. A PCR (polymerase chain reaction) product was used to replace a neutral gene with an antibiotic resistance (Ab^{R}) marker (selected) and a PCR product to introduce a nonsense point mutation into lacZ (unselected) (FIGS. 1A and 6A-6B).

FIG. 6A is a schematic diagram showing co-transformation and recombination of a bacterial genome with two selectable markers from PCR products. FIG. 6B is a schematic diagram showing the recombination of a bacterial genome with one selectable marker from a PCR product and co-transformation of a plasmid carrying the selectable marker for kanamycin resistance. FIG. 6C is a graph showing the congression or random uptake of VC1807 kanamycin resistance gene in a PCR product and plasmid kanamycin resistance gene. FIG. 6D is a graph showing the transformation efficiency of TG1 (recA+) cells and DH5α (recA−) cells.

The highest rates of co-transformation (˜50-65%) were obtained when the unselected marker had ≧2 kb arms of homology and was present at high concentrations (3 μg/mL) (FIGS. 1B and 1C). There were fewer constraints on the selected marker. As expected, increasing the length of homology or the amount of the selected marker increased the number of transformants obtained (FIGS. 1D and 1F), however, this did not substantially alter co-transformation frequency, even when the selected product was present at equimolar concentration to the unselected product (FIGS. 1E and 1G). This indicated that the increased concentration of unselected DNA in the transformation reaction and not the ratio of selected:unselected DNA was critical for optimal co-transformation. Also, distance between the selected and unselected markers on the genome did not alter co-transformation frequency as an unselected marker on a distinct chromosome exhibited a similar co-transformation frequency to the lacZ marker (FIG. 1H). Genetic edits were not limited to point mutations, as deletions and insertions of 50-1,166 bp were obtained with co-transformation frequencies of ˜60%-25%, respectively (FIGS. 1I and 1J).

### Example 2

### Assessing Bias During Natural Co-Transformation

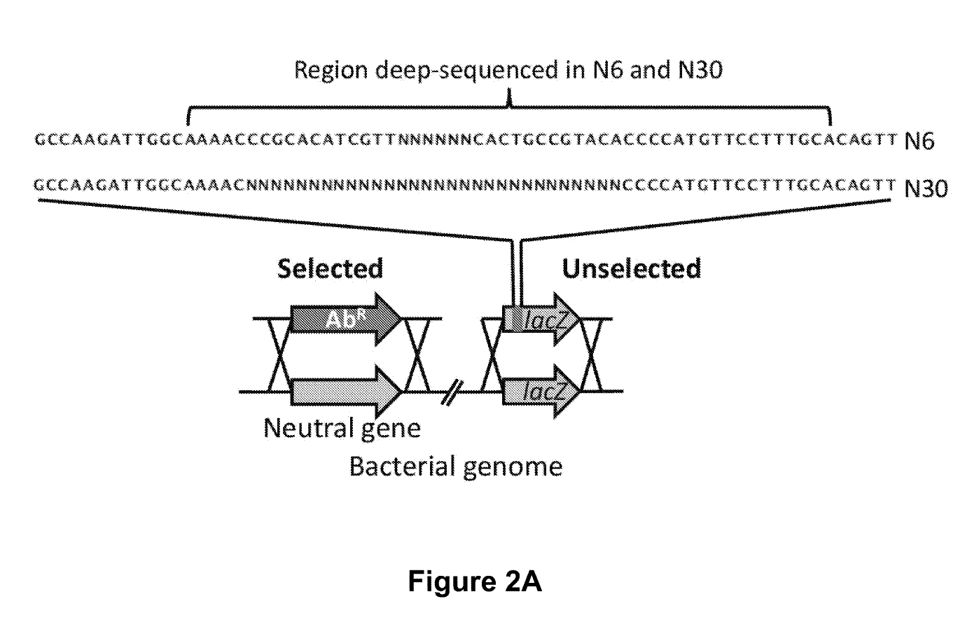

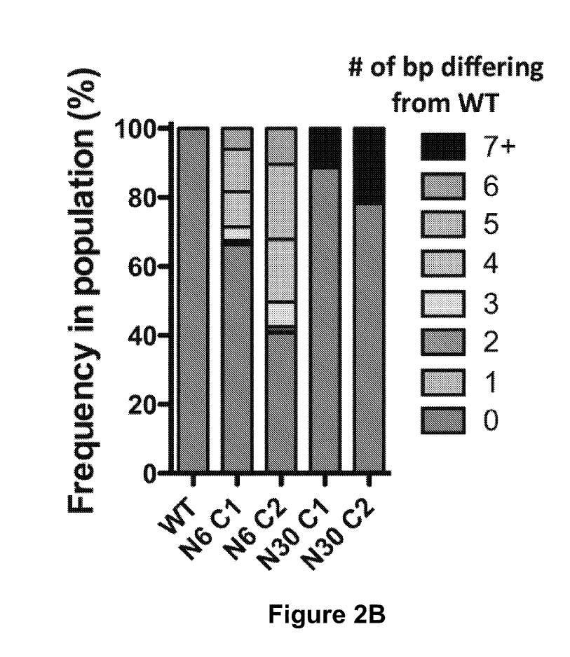

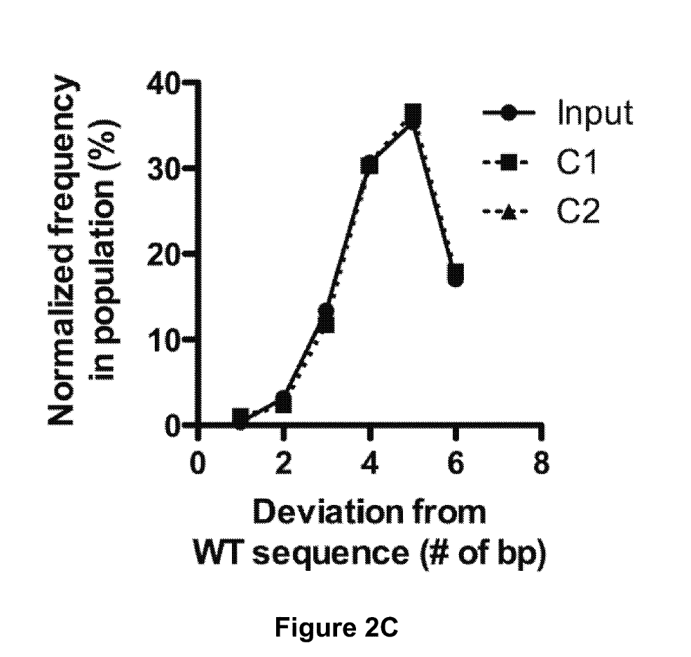

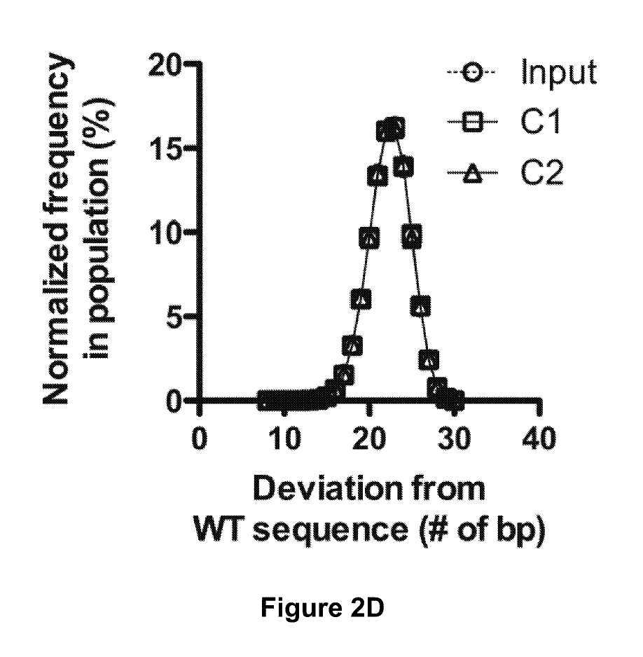

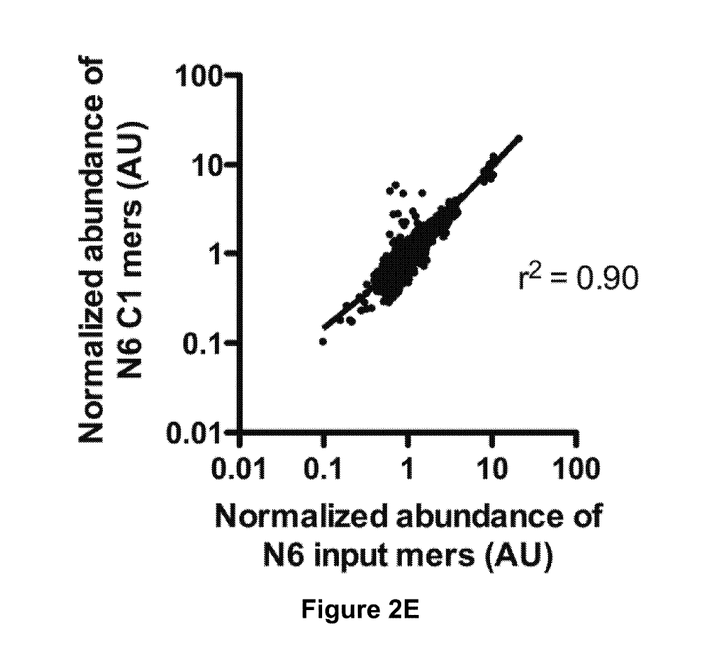

Results of co-transformation experiments show natural co-transformation can be used for unbiased directed evolution at a single genetic locus. Co-transformation experiments with PCR products were performed that had either 6 (N6) or 30 (N30) nucleotides randomized in the lacZ gene. To increase the complexity of mutations at the lacZ locus, multiple cycles of co-transformation were performed with the N6 and N30 unselected products by using selected products that alter the antibiotic resistance marker at the neutral locus at each cycle (FIGS. 2A and 2B). Based on deep sequencing of the input PCR product and output transformant pools, no increase in co-transformation frequency was found for sequences closer to the WT for either the N6 or N30 samples (FIGS. 2C and 2D). Furthermore, a significant correlation was found between the abundance of N6 mers in the input PCR pool to the output transformant pool, further supporting that there was little to no bias in the N6 mers recombined into the genome during co-transformation (FIG. 2E). Thus, these data suggest that natural co-transformation can be used for unbiased directed evolution at a single genetic locus.

### Example 3

### Multiplexed Genome Editing by Natural Transformation (MuGENT) Optimizes Natural Transformation in V. cholerae

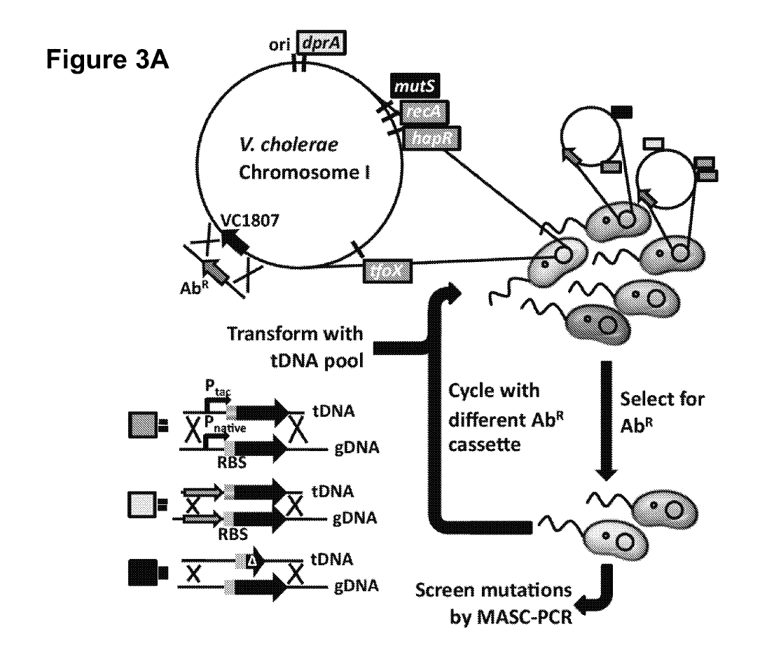

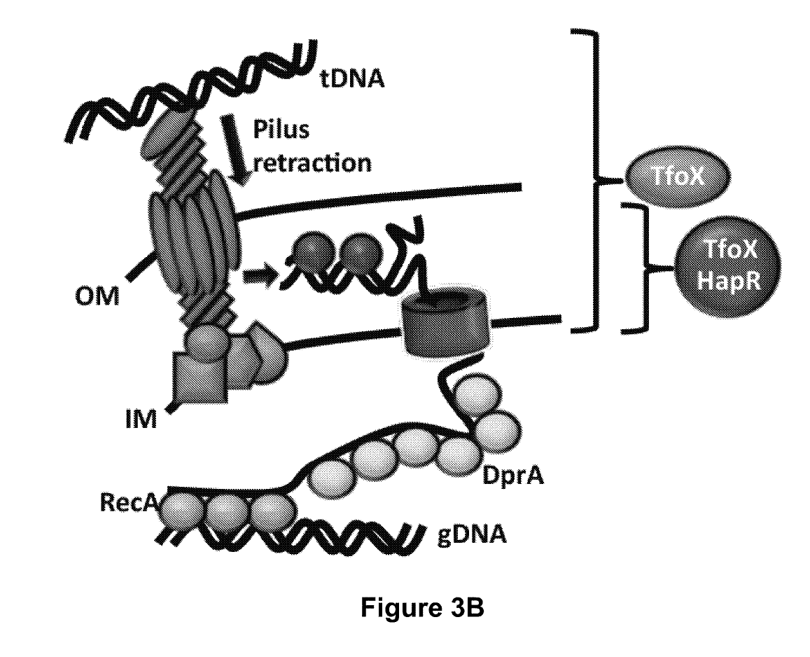

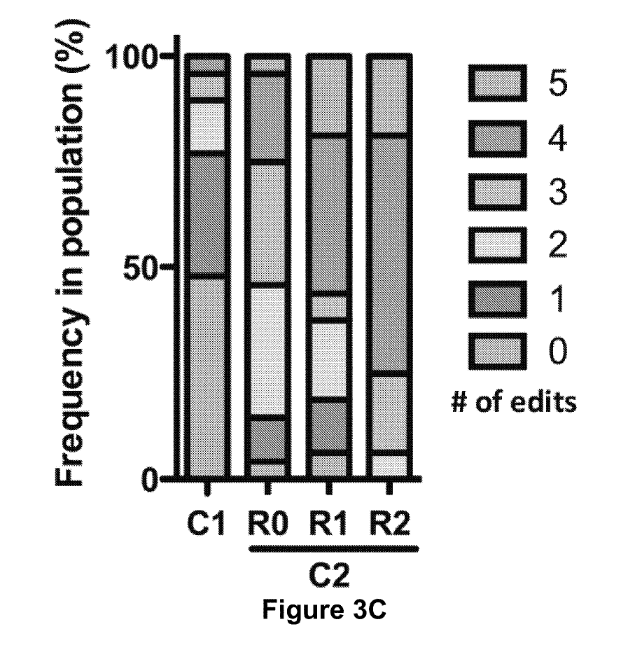

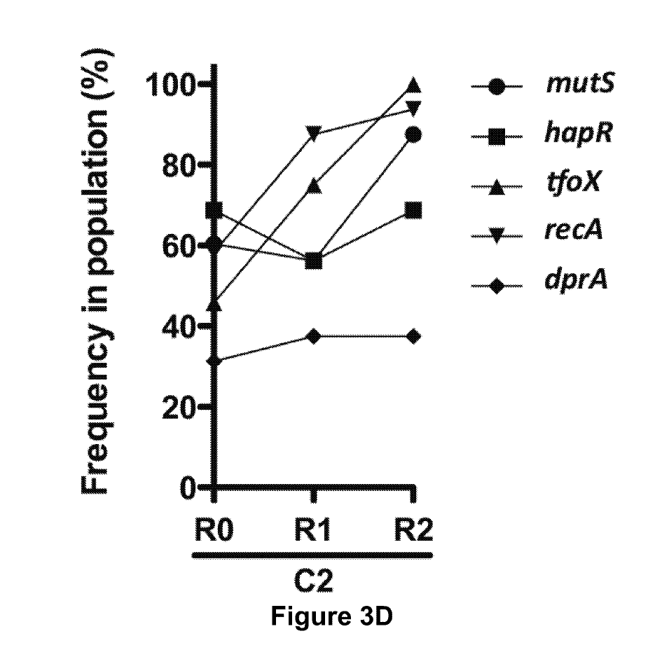

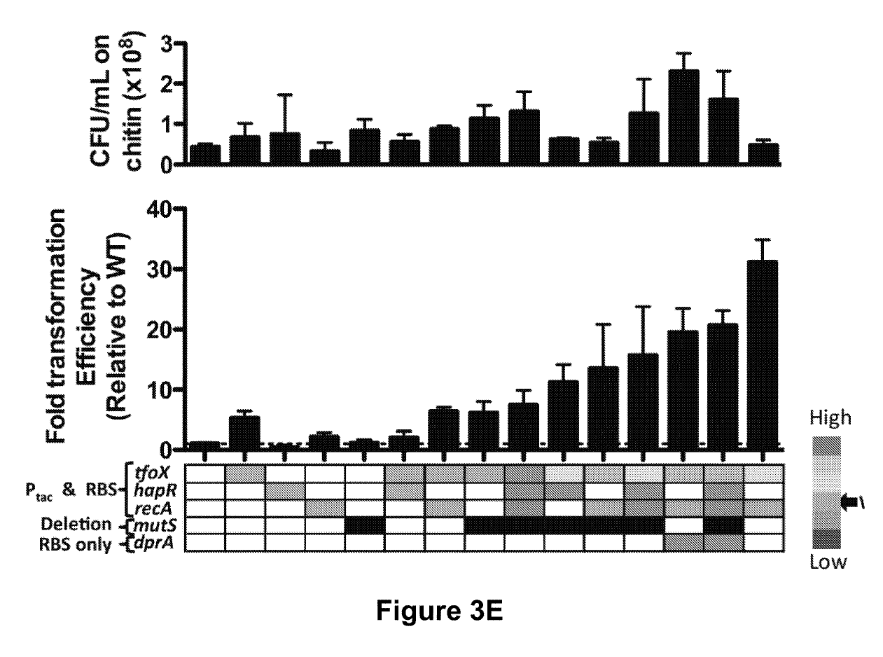

Editing genomes in multiplex in the absence of selection can be used for “accelerated evolution” to optimize metabolic pathways and phenotypes. Thus, natural co-transformation was assessed if it can be used for multiplex genome editing. Since genome edits do not require selection, output transformants can have any number of edits, and using multiple cycles of co-transformation, the complexity of gene edits were increased in the final transformant pool (FIG. 3A).

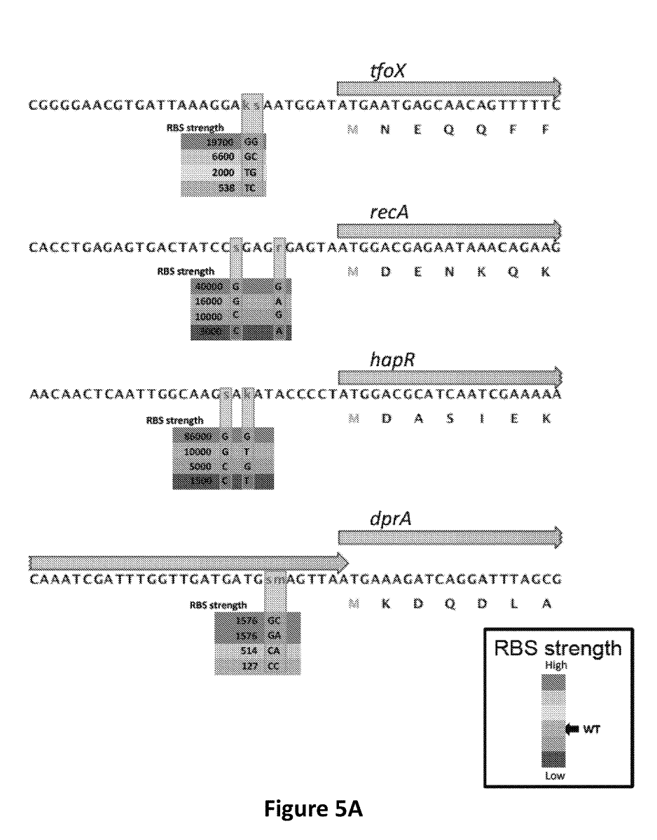

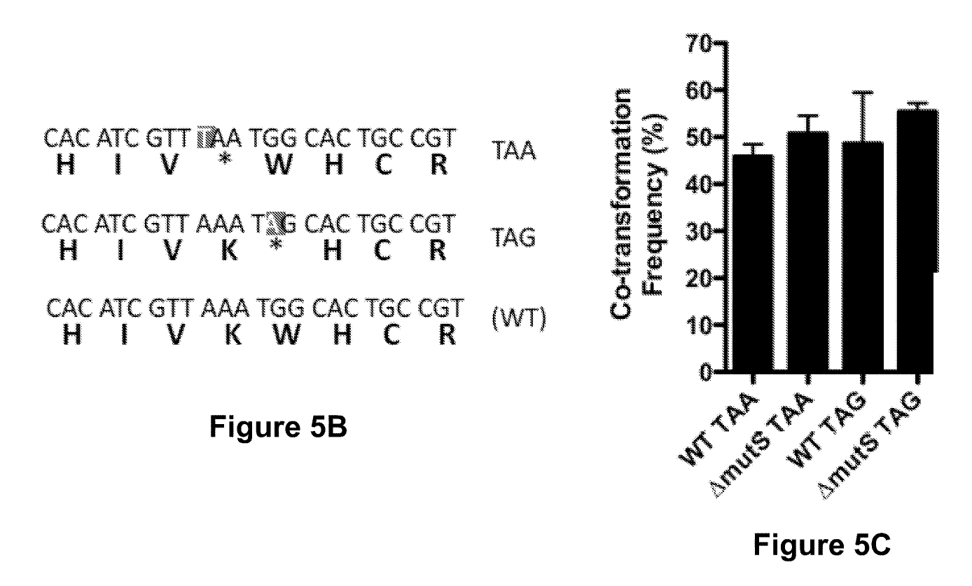

As a proof-of-concept, the phenotype of natural transformation in V. cholerae was optimized, as many of the genes involved in natural transformation and their regulation are well characterized. In this approach, the genetic loci that would impact distinct steps of natural transformation were targeted, including uptake of transforming DNA (tDNA) into the periplasm (tfoX), transport across the inner membrane (tfoX and hapR), protection of cytoplasmic single-stranded tDNA (dprA) and homology searching/integration of tDNA (recA) (FIG. 3B). The tfoX, hapR and recA genes were targeted for promoter replacement (promoter construct=LacI-inducible P_{tac} and rrnB antiterminator) and ribosome-binding site (RBS) tuning, while dprA was targeted for RBS tuning alone, as this gene is within an operon. RBS tuning was accomplished by semi-randomized mutagenesis of two key positions within the RBSs of these four genes (FIG. 5A). The mismatch repair (MMR) system can prevent or correct genetic edits subsequent to integration. Therefore, mutS was also targeted, a critical component of MMR, for inactivation. In total, there were 1000 possible combinations for these genome edits.

First, co-transformation was used to introduce genome edits into a population of cells in multiplex. PCR products for each mutation were mixed at equimolar concentrations with a selectable marker in transformation reactions. Multiple cycles of MuGENT were carried out by using selected products to alter the antibiotic resistance cassette at the neutral locus at each cycle. Transformants were screened by multiplex allele-specific colony (MASC) PCR, and after a single cycle of co-selection (C1), ˜50% of the population was found to have at least one genetic edit (FIG. 3C). After a second cycle of co-selection (C2/R0), ˜90% of the population contained at least one edit and ˜4% had edits at all five loci. Both cycles of co-selection were accomplished in under one week. Thus, MuGENT is a feasible and highly effective strategy for generating complex mutant pools within a defined set of loci.

Next, the goal was to select and characterize edited strains with the phenotype of improved natural transformation. Thus, the C2/R0 mutant pool was subjected to two additional rounds of natural transformation using only a selected marker to enrich for strains with a phenotype of increased natural transformability (R1 and R2). After these two additional rounds of enrichment, edits at tfoX and recA were in ˜100% and ˜90% of the population, respectively, suggesting that these edits enhanced natural transformation (FIG. 3D). Indeed, when the defined edited strains were tested with the wildtype RBS, the transformation efficiencies of the tfoX, recA and tfoX recA strains were greater than the parent strain (FIG. 3E). Next, seven randomly chosen colonies were isolated from the final enriched pool. All of the chosen colonies had transformation efficiencies higher than the parent strain, and many were improved compared to any defined singly and doubly edited strains (FIG. 3E, lower panel). In general, strains with edits in hapR displayed improved growth on chitin, indicating why there may have been selection for strains with 3-5 edits that included hapR after two rounds of selection (FIG. 3C), despite their having a lower transformation efficiency compared to a strain with only tfoX and recA edited (FIG. 3E).

Regardless, MuGENT allowed for the rapid isolation of multiply edited strains with improved natural transformation phenotypes, representing up to a ˜30-fold increase over the parent strain and ˜6-fold increase over any singly edited strain. This was likely attributed to the combinatorial effect of these RBS optimized genome edits. Assessing the combinatorial space explored in these experiments in a sequential manner using classic techniques would take an inordinate amount of time and effort. Thus, these experiments demonstrate that MuGENT is an excellent platform for accelerated evolution in naturally competent microbes.

### Example 4

### MuGENT Rapidly Generates all Possible Mutant Combinations of a Defined Gene Family in Streptococcus pneumoniae

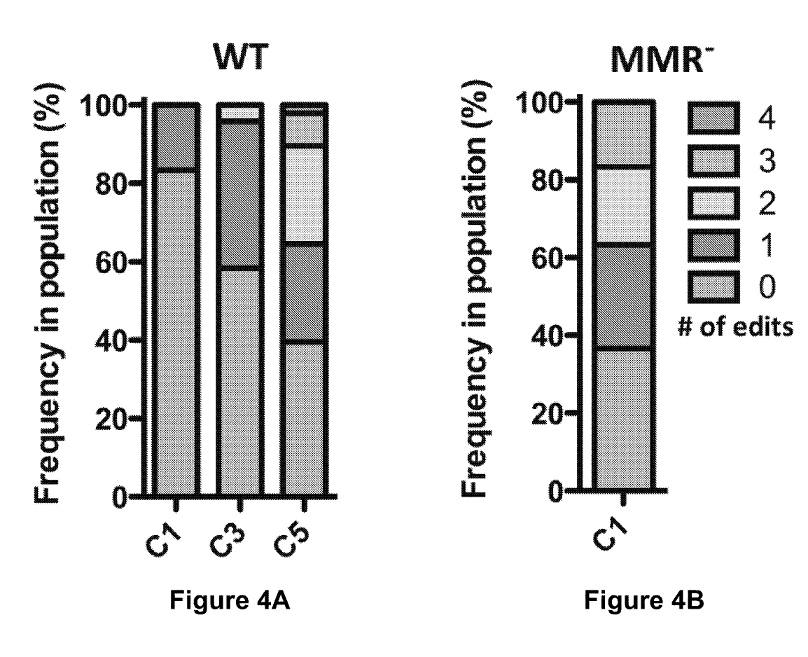

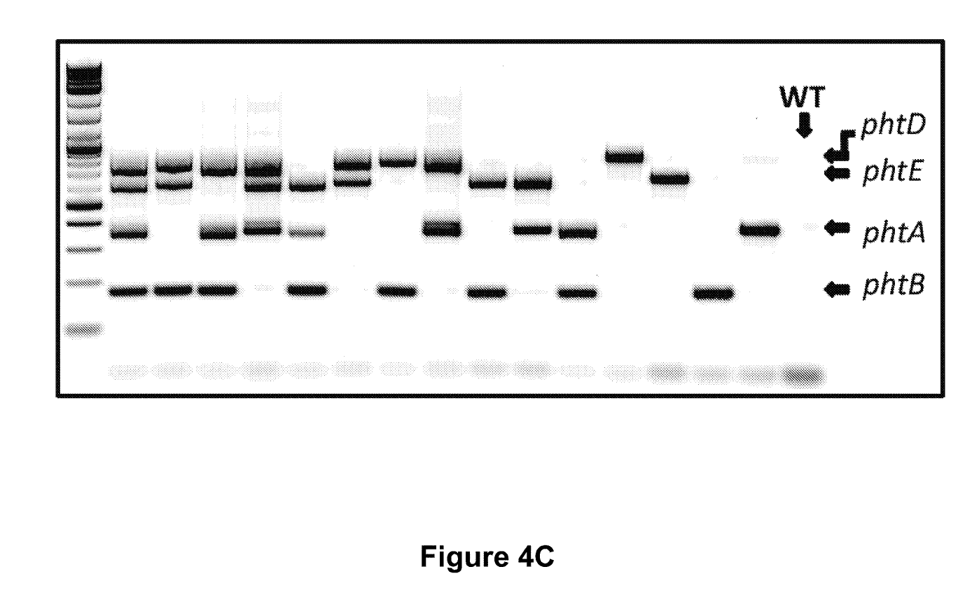

Genetic redundancy can hinder uncovering phenotypes in organisms. Using MuGENT, redundancies were revealed by generating pools of defined mutant combinations. To test this, and demonstrate MuGENT in another species, the four phi′ genes in S. pneumoniae were targeted for inactivation. These genes have previously been implicated as redundant zinc-binding proteins. Using MuGENT, premature tandem stop codons were introduced into phtA, phtB, phtD and phtE in a combinatorial fashion. Co-transformation frequency was lower in S. pneumoniae compared to V. cholerae. Despite this, after five cycles of MuGENT, which took one week to perform, all 16 possible combinations were obtained for these genome edits (FIGS. 4A and 4C). The difference in editing frequency between V. cholerae and S. pneumoniae may be due to differences in the efficacy of mismatch repair (MMR) in these bacteria. To test this, the combinatorial pht gene inactivation experiment was repeated in a strain lacking MMR, and it was found that editing frequencies were dramatically improved (FIG. 4B).

In contrast, MMR showed a minimal effect when tested in V. cholerae (FIGS. 5A and 5B). The basis for this differential effect is currently unknown. Thus, use of MMR deficient S. pneumoniae increased the speed of MuGENT, however, this may also have increased the frequency of off-target mutations in the genome. Indeed, this was observed during MAGE (multiplexed automated genome engineering), which was commonly performed in MMR deficient strains. Recently, it was demonstrated that use of temperature-sensitive MMR alleles allowed for efficient MAGE, while limiting off target mutations. Application of conditional MMR deficiency to S. pneumoniae may also allow for efficient MuGENT, while limiting off-target effects.

MuGENT can be used for multiplex genome editing in the two naturally transformable bacteria; the gram-negative V. cholerae and the gram-positive S. pneumoniae. Both of these microorganisms are human pathogens, and MuGENT has the potential to uncover novel phenotypes and provide deep insight into how these bacteria interact with their mammalian hosts. Specifically, MuGENT provides the tools necessary to rapidly generate strains with large numbers of defined mutations as well as holds the potential to uncover novel biology as a platform for genetic interaction studies.

Non-pathogenic species of Vibrio and Streptococci, however, may also benefit from MuGENT as a platform for accelerated evolution. Vibrio species are naturally found in the aquatic environment. Chitin is a food industry waste product and the most abundant biomolecule in aquatic environments, and Vibrio naturally degrade and utilize chitin as a carbon and nitrogen source. Thus, these species could be exploited for biotechnology applications using chitin as an input carbon source. Additionally, some Vibrio species, namely V. splendidus, are capable of degrading and utilizing alginate, further expanding the possible carbon sources that could be exploited for biotechnology applications. Currently, a limiting feature of these species has been a lack of the genetic tools required for efficient metabolic and phenotypic engineering. To date, natural competence and transformation has been demonstrated in a number of Vibrio species. Thus, MuGENT provides the genetic tools necessary for the development of Vibrio species for use in diverse biotechnology applications. The probiotic microbe Streptococcus thermophilus is commonly used in the dairy industry and is naturally competent. Thus, MuGENT may be used for metabolic engineering in S. thermophilus to alter or enhance its use in the dairy industry as well as enhance the probiotic activity of this species.

A large number of diverse species of microbes are known or predicted based on bioinformatics to be naturally transformable and thus would be candidates for use of MuGENT. These include, but are not limited to, species of Bacillus, Cyanobacterium, Lactococcus, Acinetobacter, Neisseria and Haemophilus. Thus, this method should be broadly applicable for diverse research and biotechnology applications.

### Example 5

### Co-Transformation Mutagenesis of a Bacterial Artificial Chromosome in a V. cholerae Host Strain

MuGENT can be used for multiplex genome editing of a bacterial artificial chromosome. Bacterial artificial chromosomes (BACs) allow for cloning of large segments of insert DNA (100 kb-350 kb) in bacteria. Once DNA is cloned into a BAC, it can be genetically engineered using the genetic tools available in bacterial systems. For this reason, BACs have been used extensively for generating transgenic animal models and for mutagenesis of large viruses (Herpesviruses, Coronaviruses, Poxviruses, and Flavoviruses).

Currently, the most common bacterial host used for maintenance of BACs is Escherichia coli, and the best method available for mutagenesis of BACs is known as “recombineering”. This method allows for mutagenesis of BACs at an efficiency of 1 in 10,000-100,000 cells (e.g. 0.01%-0.001% of cells contain the mutation). Thus, a selectable marker (i.e. an antibiotic resistance gene) is often used to isolate bacterial cells that contain the desired mutant BAC. In most instances, however, it is undesirable to have these selectable markers in the final BAC.

There are three methods that allow for BAC mutagenesis where the resultant BAC lacks a selectable marker. In the first method, there are two steps, where 1) recombineering is performed using a selectable marker that is flanked by recombinase target sites. Following selection for the mutant BAC using the selectable marker, the marker is then 2) specifically excised by expression of a site-specific recombinase. In this procedure the resultant BAC lacks a selectable marker, takes multiple steps, and contains a “scar” sequence for the recombinase target sequence. In the second method, a genetic cassette containing a selectable marker and a counter-selectable marker is used for recombineering. This method also has two steps where 1) recombineering is performed to introduce this cassette at the desired locus and selected via the selectable marker. Then 2) a second round of recombineering is performed which replaces the genetic cassette with the desired mutation, and this mutation is selected via the counter-selectable marker (i.e. select for cells which now lack the genetic cassette). Here, the resultant BAC lacks the selectable marker and is “scarless”, but requires multiple steps to obtain the edited BAC. In a third method, 1) recombineering is performed without any selectable marker and the rare mutant BAC (0.001%) is recovered 2) via enrichment of the recombineered populations. This enrichment requires many steps of dilution and PCR to isolate these rare BACs. This method allows for scarless BAC mutagenesis with a single recombineering reaction, however, this procedure requires a lengthy process to enrich for the edited BAC. Additionally, for all three of the methods described above, if multiple mutations need to be generated in these BACs they must be made sequentially (i.e. one at a time).

Here, a novel mutagenesis procedure that allows for multiplex mutagenesis of BACs in a single step is described. Results demonstrate that natural cotransformation could be used for scarless genome editing in the bacterium Vibrio cholerae. This method is based on cotransformation of two or more DNA products into a BAC. One product has a selectable marker, which would integrate at a neutral locus (e.g. replacing a dispensable gene with an antibiotic resistance marker), and the other product has a scarless mutation, which would integrate at a locus of interest. BACs used in E. coli can also be propagated in V. cholerae.

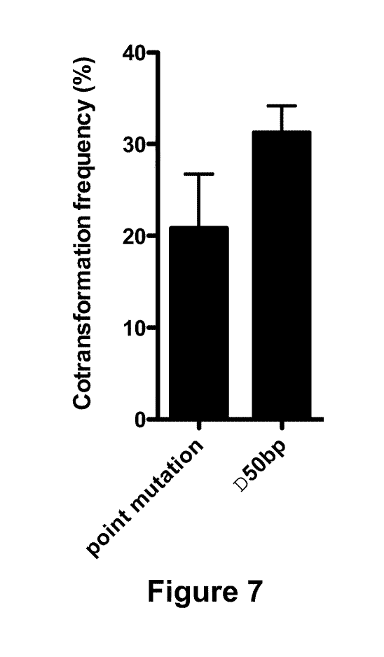

Preliminary results showed a BAC was edited with an efficiency of ˜1 in 2.5 cells (e.g. 40% of cells following this mutagenesis procedure contain the desired scarless mutation) using cotransformation in V. cholerae. FIG. 7 is a graph showing co-transformation frequency in cotransformation mutagenisis of a BAC in a V. cholerae host strain. The V. cholerae host strain had an inactivated lacZ gene, overexpresses tfoX from an IPTG (isopropyl beta-D-1-thiogalactopyranoside) inducible promoter and harbors pBluelox (a bacterial artificial chromosome vector backbone). This strain was transformed in LB (Luria broth) medium containing 100 μM IPTG with a selected marker (a PCR product that integrated into the V. cholerae chromosome and conferred resistance to spectonomycin) and an unselected product, which introduced a triple point mutation or deletes 50 bp of the lacZ gene of pBluelox. Transformants were screened for integration of the unselected product by mutation specific colony PCR. Data are from two independent biological replicates.

This novel method would lend itself to generating a BAC mutagenesis kit where a V. cholerae strain, the DNA required for selection during cotransformation and positive controls for BAC mutagenesis are supplied. The user of the kit would need only supply the BAC that needs editing and a PCR product containing the mutation of interest that will be integrated into the BAC.

The results described herein were obtained using the following methods and materials.

### Bacterial Strains and Culture Conditions

All V. cholerae and S. pneumoniae parent strains are described in Table 2. V. cholerae and S. pneumoniae were routinely grown exactly as described herein. For V. cholerae, when appropriate, media was supplemented with 50 μg/mL Kanamycin, 100 μg/mL Spectinomycin, 100 μg/mL Streptomycin or 100 μg/mL Ampicillin. For S. pneumoniae, when appropriate, media was supplemented with 200 μg/mL Spectinomycin, 4 μg/ml Chloramphenicol or 100 μg/mL Streptomycin.

TABLE 2
Parent Strains.

| Strain name in | | |
|---|---|---|
| manuscript | Genotype and antibiotic resistance(s) | Description |
| WT V. cholerae | E7946 Sm^{R} | Sm^{R} derivative of wild type V. cholerae O1 El Tor¹, which was used to |
| | | optimize co-transformation in V. cholerae. |
| Parent strain for | E7946 SmR ΔVC1807 lacI^{q}::lacZ Kan^{R} | Contains lacI^{q} in the lacZ gene, which regulates the P_{tac} promoter used in |
| V. cholerae MuGENT | | MuGENT for natural transformation in V. cholerae. Generated by co- |
| | | transformation with a selected product that replaces VC1807 with |
| | | kanamycin resistance marker. |
| ΔmutS V. cholerae | E7946 SmR ΔVC0535 (mutS) Spec^{R} | mutS gene replaced with Spec^{R} marker, used to study the effect of MMR |
| | | on co-transformation in V. cholerae. |
| WT S. pneumoniae | TIGR4 | Wild type virulent serotype 4 pneumococcal isolate², used for pht |
| | | MuGENT |
| MMR deficient | TIGR4 Sm^{R} magellan2::SP_2076 (hexA) | Mariner transposon insertion in hexA, a critical component of MMR in |
| S. pneumoniae | | S. pneumoniae³, used for pht MuGENT in an MMR deficient strain |

### Generation of Mutant Constructs and Strains

Mutant constructs for selected and unselected PCR products throughout this study were generated via splicing by overlap extension (SOE) PCR exactly as described herein using Phusion polymerase, as this enzyme has a low error rate compared to other PCR polymerases (Thermo Scientific). The primers used to generate all SOE products are listed in Table 3. In V. cholerae, the neutral locus targeted with the selected product was VC1807, a transposase pseudogene with an authentic frameshift, which was replaced with a Spectinomycin, Kanamycin or Ampicillin resistance marker. In S. pneumoniae, the selected product replaced SP_1051 with a Chloramphenicol or Spectinomycin resistance marker. The promoter construct consisting of P_{tac} and the rrnB antiterminator used during MuGENT in V. cholerae was derived from the end of a previously described Tn10 transposon.

TABLE 3
List of Primers.

| | | |
|---|---|---|
| Primer | | |
| Name | Sequence (5′ → 3′) | Description |

Mutant construct primers

| | | |
|---|---|---|
| ABD346 | GTAGAATAAGTGCGGCGTTGAGCC | F1 oligo for 3 kb UP arm ΔVC1807 SOE |
| ABD344 | GATTAGCAACGATTCTAGCGCAGGAG | F1 oligo for 2Kb UP arm ΔVC1807 SOE |
| ABD342 | ATTTTTCAGTTGGCCTACAATGCTTTCC | F1 oligo for 1Kb UP arm ΔVC1807 SOE |
| ABD340 | gtcgacggatccccggaatACGTTTCATTAGTCACCTCTATTGTTAACTTGTTC | R1 oligo for UP arm ΔVC1807 SOE |
| ABD341 | gaagcagctccagcctacaTAGTCGAAAATAAAAAAAAGAGGCTCGCCTC | F2 oligo for DOWN arm ΔVC1807 SOE |
| ABD343 | CGATGAGGATAAAAAACACGTACAGGCC | R2 oligo for 1Kb DOWN arm ΔVC1807 SOE |
| ABD345 | CTTGCTAACCGTTGGTGTTACCAGC | R2 oligo for 2Kb DOWN arm ΔVC1807 SOE |
| ABD347 | GCGCCCAATGTTGTCCCTTTGATG | R2 oligo for 3Kb DOWN arm ΔVC1807 SOE |
| ABD123 | ATTCCGGGGATCCGTCGAC | F oligo for Kan^{R}, Amp^{R}, and Spec^{R} markers in V. cholerae |
| ABD124 | TGTAGGCTGGAGCTGCTTC | R oligo for Kan^{R}, Amp^{R}, and Spec^{R} markers in V. cholerae |
| ABD357 | GCGCTGCCAAGTAGGTGTAACG | F1 oligo for UP arm of ΔmutS SOE for V. cholerae |
| ABD358 | gtcgacggatccccggaatCATAATCTTATGTCGCTGCTTATCATCATCTG | R1 oligo for UP arm of ΔmutS SOE for V. cholerae |
| ABD359 | gaagcagctccagcctacaTAGTTATTGCCCATATCTCAAGCATGGAATC | F2 oligo for UP arm of ΔmutS SOE for V. cholerae |
| ABD360 | AGATCTTGCCTGATGACGCTTTACTC | R2 oligo for UP arm of ΔmutS SOE for V. cholerae |
| ABD336 | ACCCTAAGCGGTTCAATTTTGTCGATG | F1 oligo for 3 kb lacZ UP arm |
| ABD334 | AGTGCTCCGGACTCTTTGCTCTG | F1 oligo for 2 kb lacZ UP arm |
| ABD332 | GGCTGAACGTGGTTGTCGAAAATGAC | F1 oligo for 1 kb lacZ UP arm |
| ABD333 | ATCACATGCCCCAATTCGGGATG | R2 oligo for 1 kb lacZ DOWN arm |
| ABD335 | CACTGCTCACTAGCGATGCAGTG | R2 oligo for 2 kb lacZ DOWN arm |
| ABD337 | TGATCCGATGATCTTTTCGCCCACC | R2 oligo for 3 kb lacZ DOWN arm |
| ABD329 | GAACATGGGGTGTACGGCAGTGCCATTaAACGATGTGCGGGTTTTGCCAATC | R1 oh go go for lacZ TAA transversion mutation |
| | TTG | |
| ABD328 | CAAGATTGGCAAAACCCGCACATCGTTtAATGGCACTGCCGTACACCCCATG | F2 oligo for lacZ TAA transversion mutation |
| | TTC | |
| ABD331 | GAACATGGGGTGTACGGCAGTGCtATTTAACGATGTGCGGGTTTTGCCAATC | R1 oligo for lacZ TAG transversion mutation |
| | TTG | |
| ABD330 | CAAGATTGGCAAAACCCGCACATCGTTAAATaGCACTGCCGTACACCCCAT | F2 oligo for lacZ TAG transversion mutation |
| | GTTC | |
| ABD361 | CCTCtctccctatagtgagtcgtattaatttcATTTAACGATGTGCGGGTTTTGCCAATC | R1 oligo for Δ50, Δ100, Δ464, and Δ1166 bp in lacZ |
| ABD362 | CGTTAAATgaaattaatacgactcactatagggagaGAGGCTCGTTTGGATGTTGGG | F2 oligo for Δ50 bp in lacZ |
| ABD363 | CGTTAAATgaaattaatacgactcactatagggagaGaGGTTTGCTCTGTTTGAGAAGCC | F2 oligo for Δ100 bp in lacZ |
| ABD364 | ATCGTTAAATgaaattaatacgactcactatagggagaGAGGGTGAAAACCTGTTGGTGG | F2 oligo for Δ464 bp in lacZ |
| ABD365 | CATCGTTAAATgaaattaatacgactcactatagggagaGAGGCCAATCTCGAAACCCAC | F2 oligo for Δ1166 bp in lacZ |
| ABD288 | GCCAATCGTGGAGTCGACCC | F1 oligo for a transversion point mutation upstream of |
| | | VCA0063 that abolishes an MboI cleavage site |
| ABD069 | AAATAAaATCAATTATCATTTGTGTTATTTTAGAGC | R1 oligo for a transversion point mutation upstream of |
| | | VCA0063 that abolishes an MboI cleavage site |
| ABD068 | AAATGATAATTGATtTTATTTAGATTTGTGTTAGAGTGGCGAC | F2 oligo for a transversion point mutation upstream of |
| | | VCA0063 that abolishes an MboI cleavage site |
| ABD289 | CGTCACCTGAAGATTCCATCCGTC | R2 oligo for a transversion point mutation upstream of |
| | | VCA0063 that abolishes an MboI cleavage site |
| ABD841 | GGCACTGCCGTACACCCCATGTTCCGGTACCCGACACCATCGAATG | F oligo to amplify lac1^{q} with overlaps to insert into the lacZ |
| | | gene of V cholerae. |
| ABD842 | GGCTCTCTGGCTTATTGTGGGGGGAAACCTGTCGTGCCAGCTGC | R oligo to amplify lac1^{q} with overlaps to insert into the lacZ |
| | | gene of V cholerae. |
| ABD840 | TTAATTGCGTTGCGCTCACTGCCCGACTCCCGTTCTGGATAATGTTTTTTGC | F oligo for Pta, and rrnB promoter construct (i.e. the |
| | | MIDDLE for the MuGENT PCR products of tfoX, hapR, and |
| | | recA) |
| ABD625 | CTGATGAATCCCCTAATGATTTTGG | R oligo for Ptac and rrnB promoter construct (i.e. the |
| | | MIDDLE for the MuGENT PCR products of tfoX, hapR, and |
| | | recA) |
| ABD808 | GTGTGGCTTGTCGGTCGGCAAAGG | F1 oligo for UP arm of tfoXMuGENT SOE product |
| ABD809 | CGGGCAGTGAGCGCAACGCAATTAAATGCAATACTTTTGCGCCAGATTATG | R1 oligo for UP arm of tfoX MuGENT SOE product |
| ABD810 | CAAAATCATTAGGGGATTCATCAGGGGGAACGTGATTAAAGGAksAATGGA | |
| | TATGAATG | F2 oligo for DOWN arm of tfoX MuGENT SOE product |
| ABD811 | GTTAGAAGAGCTTATCCATCACCG | R2 oligo for DOWN arm of tfoX MuGENT SOE product |
| ABD812 | AAATGGAGTTTGATCGCATTGGC | F1 oligo for UP arm of recA MuGENT SOE product |
| ABD813 | CGGGCAGTGAGCGCAACGCAATTAACCCCTGTATAGAAAAAAGTTTGGC | R1 oligo for UP arm of recA MuGENT SOE product |
| ABD814 | CAAAATCATTAGGGGATTCATCAGGTGACTATCCSGAGRGAGTAATGGAC | F2 oligo for DOWN arm of recA MuGENT SOE product |
| | GAGAATAAAC | |
| ABD815 | TGATCAGCGTTTGGAATACGTCG | R2 oligo for DOWN arm of recA MuGENT SOE product |
| ABD816 | AAAGCACCAAATAATCCCGCGACC | F1 oligo for UP arm of hapR MuGENT SOE product |
| ABD817 | CGGGCAGTGAGCGCAACGCAATTAACTCTGTTGGGTGAAAAATGTGC | R1 oligo for UP arm of hapR MuGENT SOE product |
| ABD818 | CAAAATCATTAGGGGATTCATCAGATTGGCAAGSAKATACCCCTATGGACG | F2 oligo for DOWN arm of hapR MuGENT SOE product |
| | CATCAATCG | |
| ABD819 | GGCCTGCTTAGCAAATTTACCAG | R2 oligo for DOWN arm of hapR MuGENT SOE product |
| ABD820 | CGCTCTTATCTGCTTGGATAATGG | F1 oligo for UP arm of dprA MuGENT SOE product |
| ABD821 | GCCGCTAAATCCTGATCTTTCATTAACTKSCATCATCAACCAAATCGATTTG | R1 oligo for UP arm of dprA MuGENT SOE product |
| | TTC | |
| ABD822 | AGTTAATGAAAGATCAGGATTTAGCGGC | F2 oligo for DOWN arm of dprA MuGENT SOE product |
| ABD823 | TGAAGTACAAGGCCAGTTACTGG | R2 oligo for DOWN arm of dprA MuGENT SOE product |
| ABD824 | TTTAGCCCCATTGGCGAACTGGG | F1 oligo for UP arm of mutS MuGENT SOE product |
| ABD825 | GAGTATCTTTGACGTATTGGATCtcatattatactaCATAATCTTATGTCGCTGCTTA | R1 oligo for UP arm of mutS MuGENT SOE product |
| | TC | |
| ABD826 | GATAAGCAGCGACATAAGATTATGtagtataatatgaGATCCAATACGTCAAAGAT | F2 oligo for DOWN arm of mutS MuGENT SOE product |
| | ACTC | |
| ABD360 | AGATCTTGCCTGATGACGCTTTACTC | R2 oligo for DOWN arm of mutS MuGENT SOE product |
| Spn cat F | CGGTATCGATAAGCTTGATG | F oligo for SpecR and CmR markers in S. pneumoniae |
| Spn cat R | TGGAGCTGTAATATAAAAACCTTCTTC | R oligo for SpecR and CmR markers in S. pneumoniae |
| ABD528 | GGAGAACTTAAATGAATTGTAGAGGACATG | F1 oligo for UP arm of ΔSP_1051 SOE |
| 1051_R1 | CATCAAGCTTATCGATACCGTTCCATTAGGCCACCATC | R1 oligo for UP arm of ΔSP_1051 SPE |
| 1051_F2 | GAAGAAGGTTTTTATATTACAGCTCCAGGGGGAAAAGAGACTTAATG | F2 oligo for DOWN arm of ΔSP_1051 SOE |
| ABD529 | CTATGTGTTCAGCTGACTCCCATG | R2 oligo for DOWN arm of ΔSP_1051 SOE |
| ABD534 | AAAAGTGCACGACAAATAGCCC | F1 oligo for phtD inactivation MuGENT SOE product |
| ABD535 | GACAATGTCTGAATCCTaCAACTGcTAATTCGGATCTTTCATGAGGAGC | R1 oligo for phtD inactivation MuGENT SOE product |
| ABD536 | GCTCCTCATGAAAGATCCGAATTAgCAGTTGtAGGATTCAGACATTGTC | F2 oligo for phtD inactivation MuGENT SOE product |
| ABD539 | GACAATATCAGCGTCTTaAAGTTGcTAGTTTGGATCCTTCATCAAGAGTTCTT | R2 oligo for phtD inactivation MuGENT SOE product |
| | CAC | |
| ABD536 | GCTCCTCATGAAAGATCCGAATTAgCAGTTGtAGGATTCAGACATTGTC | F1 oligo for phtE inactivation MuGENT SOE product |
| ABD539 | GACAATATCAGCGTCTTaAAGTTGcTAGTTTGGATCCTTCATCAAGAGTTCTT | R1 oligo for phtE inactivation MuGENT SOE product |
| | CAC | |
| ABD540 | GTGAAGAACTCTTGATGAAGGATCCAAACTAgCAACTTtAAGACGCTGATAT | F2 oligo for phtE inactivation MuGENT SOE product |
| | TGTC | |
| ABD541 | AAAGGATAACAACAGATCCAGCTGC | R2 oligo for phtE inactivation MuGENT SOE product |
| ABD548 | GTTAATGAGGTCAAGGGTGGATAgGTTATCtAGGTAGATGGAAAATACTATG | F1 oligo for phtE inactivation MuGENT SOE product |
| ABD543 | GACAATGTCTGAATCCTaCAACTGcTAATTCGGATCTTTCATGAGGAGC | R1 oligo for phtE inactivation MuGENT SOE product |
| ABD544 | GCTCCTCATGAAAGATCCGAATTAgCAGTTGtAGGATTCAGACATTGTC | F2 oligo for phtE inactivation MuGENT SOE product |
| ABD545 | TGCATAAAGCAATCCCTTTCTGATTT | R2 oligo for phtE inactivation MuGENT SOE product |
| ABD546 | GAGACATTATTACTGTCCAAGAAACGC | F1 oligo for phtE inactivation MuGENT SOE product |
| ABD547 | CATAGTATTTTCCATCTACCTaGATAACcTATCCACCCTTGACCTCATTAAC | R1 oligo for phtE inactivation MuGENT SOE product |
| ABD548 | GTTAATGAGGTCAAGGGTGGATAgGTTATCtAGGTAGATGGAAAATACTATG | F2 oligo for phtE inactivation MuGENT SOE product |
| ABD543 | GACAATGTCTGAATCCTaCAACTGcTAATTCGGATCTTTCATGAGGAGC | R2 oligo for phtE inactivation MuGENT SOE product |

MASC PCR primers

| | | |
|---|---|---|
| OLJ363 | GTGTGGGCACTCGACATATGACAAG | F oligo for tfoX, hapR, and recA |
| ABD845 | CTGAATCGAGCGTTCAATAATCG | R oligo for tfoX = 407 bp product |
| ABD846 | CATAAACATGTTTCTGATCAGCAG | R oligo for hapR = 542 bp product |
| ABD847 | TTCAGGGCCGAAGATCTCAAC | R olgo for recA = 306 bp product |
| ABD830 | GATTTGGTTGATGATGta | F1 oligo for dprA |
| ABD831 | CGATTTGGTTGATGATag | F2 oligo for dprA |
| ABD849 | GCCGCTTGAGTGGTTAAAAACACC | R oligo for dprA = 205 bp |
| ABD833 | GCGACATAAGATTATGtagtataatatg | F oligo for mutS |
| ABD848 | AGGGTATCAATGCCGTGACG | R oligo for mutS = 704 bp |
| ABD553 | AGATCCGAATTAgCAGTTat | F oligo for phtD and phtB |
| ABD569 | AGCTGGTTGAGGATTTGGTGC | R oligo for phtD = 811 bp |
| ABD618 | GCATTATCTGCTCTTGAGTT | R oligo for phtB = 194 bp |
| ABD607 | GATCCAAACTAgCAACTct | F oligo for phtE |
| ABD617 | GGCAGAAAGCTTGCTGTAAG | R oligo for phtE = 646 bp |
| ABD562 | TCAAGGGTGGATAgGTTATtt | F oligo for phtA |
| ABD619 | TTTTGTCGGCGATAGGTTC | R oligo for phtA = 382 bp |

N6 and N30 library preparation primers

| | | |
|---|---|---|
| ABD419 | CTTATGCGCAACTTCTCCGATATTCTTCTTAGCCAAGATTGGC | F oligo for PCR1, and also the sequencing oligo used for |
| | | deep-sequencing |
| ABD408 | GTGACTGGAGTTCAGACGTGTGCTCTTCCGATCTCCTTGCATCTGCCAGTTA | R oligo for PCR1 |
| | CTCGG | |
| ABD420 | AATGATACGGCGACCACCGAGATCTACACTCTTTCTTATGCGCAACTTCTCC | F oligo for PCR2 |
| | GATATTCTTCTTAGCC | |
| BC33- | CAAGCAGAAGACGGCATACGAGATNNNNNNGTGACTGGAGTTCAGACGTG | R oligo for PCR2 (N6 region indicates the barcode sequence |
| BC44 | TGCTCTTCCGATCT | that is unique for each R oligo used) |

Natural Transformation and MuGENT in V. cholerae

Natural transformation of V. cholerae following growth on chitin from shrimp shells was done as described herein. Briefly, 10⁸ CFUs (colony forming units) of mid-exponential growth phase V. cholerae were added to 80 mg of chitin flakes in 1 ml of defined artificial seawater (7 g/L). The cultures were incubated statically at 30° C. for 16-24 hours to induce natural competence. Next, the supernatant was gently removed and replaced with fresh artificial seawater to reduce the presence of DNases naturally secreted by V. cholerae. DNA was then added at the indicated concentration and incubated statically for an additional 16 hours at 30° C. To assess transformation efficiencies and biomass on chitin, reactions were directly plated onto media selective for the Ab^{R} marker (i.e. transformants) and onto media lacking antibiotics to assess total viable CFUs (i.e. total biomass on chitin). Transformation efficiency was defined as:

### CFUs of Transformants/Total Viable CFUs.

For co-transformations into lacZ, cells were plated on media selective for the Ab^{R} marker and containing 40 μg/mL 5-bromo-4-chloro-3-indolyl-D-galactopyranoside (X-gal) to assess co-transformation frequency.

For MuGENT, all PCR products, including the selected marker were added to transformation reactions at 3 μg/mL and had 3 kb arms of homology, as this was found to be the optimal length of homology and concentration for co-transformation. Under these condition, each cycle of MuGENT in a 1 mL reaction generated ≧10⁵ transformants. After reactions were incubated with DNA, samples were outgrown for 1 hr in LB broth in the absence of antibiotics. A small aliquot of the reaction (˜ 1/10^{th}) was plated to assess transformation efficiency, and single colonies from selective plates were used for MASC PCR. The remainder of each transformation was inoculated into 50 mLs of LB broth containing the appropriate antibiotic to select for transformants and grown overnight at 37° C. with aeration. The following day, this culture was diluted 1:100 in media lacking antibiotics and grown to an OD₆₀₀≈1.0. These cells were then washed and ˜10⁸ CFUs were placed onto chitin to repeat another cycle of MuGENT or to select for transformants from the mutant pool. After the first cycle of MuGENT, all subsequent transformations with this mutant pool were performed in the presence of 10 μM IPTG to induce expression of the P_{tac} promoter used in some genome edits. Growth in LB was always performed in the absence of IPTG, as IPTG-induced expression of the edited gene hapR resulted in a growth defect.

Natural Transformation and MuGENT in S. pneumoniae

Natural transformation of S. pneumoniae was performed exactly as described herein. Briefly, bacteria were grown in transformation medium (THY broth containing 13 mM HCl and 0.05% glycine) from a starting OD₆₀₀=0.02 to an OD₆₀₀=0.06. 500 μl of culture was then added to 500 μl of pre-warmed THY in glass tubes. Then, 10 μl of NaOH (1N stock), 25 μl of BSA (8% stock), 1 μl CaCl2 (1M stock) and 1.6 μl CSP 2 (350 ng/μl stock) were added to reactions in the indicated order. Reactions were then incubated for exactly 14 minutes at 37° C. prior to the addition of transforming DNA. For MuGENT, 1.5 μg of each unselected product and 300 ng of the selected product were added to a 1 mL transformation reaction. All unselected products had 2.5-3 kb arms of homology, while the selected product had 1.5 kb arms of homology. After the addition of DNA, reactions were incubated at 37° C. in a 5% CO₂ incubator for 1 hr. A small aliquot of each reaction (˜ 1/10^{th}) was then plated to assess transformation efficiency, and single colonies from selective plates were used for MASC PCR. The remainder of the transformation was plated for single colonies on media selective for transformants. The following day, these plates were flooded with THY medium to resuspend colonies. This bacterial slurry was then diluted to an OD₆₀₀=0.05 into 10 mLs of fresh THY medium and grown to an OD₆₀₀≈0.6. Cells were then washed, diluted and re-transformed to perform additional cycles of MuGENT.

### MASC PCR

At each cycle of MuGENT, 24-48 single colonies were assessed for genome edits by MASC PCR essentially as described herein. All oligos used for MASC PCR are in Table 3.

### Analysis of High-Throughput Sequencing Data for Assessing Bias During Natural Co-Transformation

After co-transformation of PCR products that randomized six (N6) and 30 (N30) bases in the lacZ gene of V. cholerae, libraries were generated for deep sequencing from genomic DNA purified from output transformant pools, as well as from the input PCR splicing by overlap extension (SOE) products. This was accomplished by first PCR amplifying with ABD419 and ABD408. This PCR was then used as the template for a second round of PCR using ABD420 and a reverse primer, which adds a unique 6 bp barcode sequence that was used to distinguish samples run together on a single lane of the Illumina HiSeq. All primers used for preparing sequencing libraries can be found in Table 3.

After sequencing, data were analyzed on the Tufts University Galaxy server. First, the “trim” tool was used to remove the first six bases for N30 samples, or 17 bases for N6 samples. Then, the clip tool was used to remove the constant sequence at the 3′ end of all molecules (N6=5′-CACTGCCGTACACCCCATGTTCCTTTGC-3′ and N30=5′-CCCCATGTTCCTTTGC-3′). Filter fastq was used to obtain reads of a length of six bases (N6) or 30 bases (N30), and a minimum quality score of 34 (on a scale of 0-41). To define the distribution of these reads in reference to how they deviate from the WT consensus, barcode splitter tool using the WT sequence as a reference was used and allowed for any number (n=1, 2, 3, . . . 30) of mismatches to define the distribution of sequences that were 1, 2, 3, etc., bases different from the WT sequence. To define the exact abundance of each N6-mer in the input and output transformant pools, the barcode splitter tool was used with the sequence of each N6-mer as a reference and allowed for 0 mismatches.

### Co-Transformation Protocol for Vibrio Cholerae

V. cholerae culture was grown overnight at 30 C in rollerdrum and shaken. Subcultures of 20 uL were transferred to 5 mL fresh LB the next morning and allowed to grow at 30 C until an OD₆₀₀=0.4-1.0 was reached. Cells in 1 mL aliquots were then pelleted at 18000ref for 1 mins (microfuge) and the supernatant removed. Cells were washed once with equal volume 0.5× instant ocean (IO) (7 g/L) and then resuspended to an OD₆₀₀=1.0 in 0.5×IO. Then, 900 uL 0.5×IO was taken and placed onto 50 mg chitin (shrimp: Sigma-C7170) for each transformation reaction. Chitin (dry) was autoclaved in 2 mL tubes beforehand. Then 100 uL washed cells from step 4 were added to each tube and vortexed to mix. Cells were then placed at 30 C for 16-24 hours static.

To minimize the exo- and endo-nuclease activities, ˜500 uL of supernatant was removed without disturbing the settled chitin. This was replaced with 300 uL fresh 0.5×IO. Then, 3-5 ug unselected PCR product was added and then selected DNA was added. For plasmids, 1 ug yielded ˜10⁴ transformants with pBAD18Kan (plasmid was prepared in a recA+ host strain (i.e. TG1)). For PCR products 100 ng yielded ˜10³-10⁴ transformants and 3 ug yielded ˜10⁵ transformants. Longer arms of homology yielded more transformants. Reactions were then inverted gently 2-3 times to mix the reactions.

The reactions were placed back at 30° C. static and the cells were allowed to incubate for 16-24 hours. Transformation reactions were vortexed vigorously and then 500 uL was transferred to 2 mL eppendorf tube containing 1 mL LB. Reactions were outgrown at 37 C with shaking for 1-3 hrs to resolve and segregate mutations as well as break up any clumps of bacteria to ensure that each colony was clonal.

Cultures were then plated on media with antibiotic to select for the selected marker and placed at 30° C. overnight. Colonies were picked and grown in 200 uL broth with antibiotic (96-well plate) and simultaneously colonies were screened for mutation by colony PCR. (i.e. a colony was picked with a sterile tip and lightly dabbed into 200 uL of selective media and the rest of the colony smashed into 50 uL water, the latter boiled and 2-3 uL used for 25 uL colony PCRs with Taq polymerase). Reactions were then placed in 96-well plate at 37° C. static. Positive wells (i.e. those containing the mutation of interest) were re-streaked for single colonies on selective media again and the genotype of a single colony from this re-streak was reconfirmed.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of introducing nucleic acid molecules into one or more cells in parallel comprising:
(a) contacting naturally competent cells with two or more nucleic acid molecules, wherein at least one of the nucleic acid sequences comprises a selectable marker; and
(b) selecting for that marker.

2. A method of introducing nucleic acid molecules into one or more cells in parallel comprising:
(a) incubating naturally competent cells under static conditions;
(b) contacting the cells with two or more nucleic acid molecules, wherein at least one of the nucleic acid sequences comprises a selectable marker; and
(c) selecting for that marker.

3. The method of claim 1, wherein the naturally competent cells are bacterial cells.

4. The method of claim 1, wherein the naturally competent cells are gram negative or gram positive.

5-6. (canceled)

7. The method of claim 1, wherein the naturally competent cells are V. cholerae or S. pneumoniae.

8. (canceled)

9. The method of claim 1, wherein at least one of the nucleic acid molecules comprises at least one arm of homology to a genetic locus of a genome of the naturally competent cells.

10-11. (canceled)

12. The method of claim 11, wherein the genome edit is introduced into a gene involved in natural transformation.

13-18. (canceled)

19. A method of introducing nucleic acid molecules into one or more polynucleotide targets in parallel comprising:
(a) contacting the polynucleotide target with two or more nucleic acid molecules, wherein at least one of the nucleic acid sequences comprises a selectable marker; and
(b) selecting for that marker.

20. The method of claim 1, wherein the nucleic acid molecules integrate at a neutral locus.

21-25. (canceled)

26. The method of claim 24, wherein the cell is a bacterial cell, yeast cell, or mammalian cell.

27. A method for optimizing the transformation efficiency of a naturally competent cell, the method comprising introducing a genetic mutation into a tfoX, recA and/or tfoX gene of the cell.

28-29. (canceled)

30. A heterogenic pool of co-transformed cells comprising:
two or more co-transformed nucleic acid molecules,
wherein the cells are naturally competent and co-transformed with the two or more nucleic acid molecules, and wherein at least one of the nucleic acid molecules comprises a selectable marker.

31. The heterogenic pool of claim 30, wherein the selectable marker is a reporter gene or a drug resistance gene.

32. The heterogenic pool of claim 30, wherein the drug resistance gene is selected from the group consisting of kanamycin resistance gene, spectinomycin resistance gene, streptomycin resistance gene, chloramphenicol resistance gene, tetracycline resistance gene, and penicillin resistance gene.

33. The heterogenic pool of claim 30, wherein the naturally competent cells are selected from the group consisting of Firmicutes, Chroococcales, Bacteriodia, Chlorobi, Deinococci, Actinobacteria, Proteobacteria, and Euryarchaeota.

34. The heterogenic pool of claim 30, wherein the naturally competent cells are selected from the species listed in Table 1.

35. The heterogenic pool of claim 30, wherein the two or more nucleic acid molecules comprise unlinked genetic markers.

36. The heterogenic pool of claim 30, wherein at least one of the nucleic acid molecules comprises at least one arm of homology to a genetic locus of a genome of the naturally competent cells.

37. The heterogenic pool of claim 30, wherein the arm of homology has a length of less than about 4 kb.

38. The heterogenic pool of claim 30, wherein at least one of the nucleic acid molecules comprises at least one genome edit.

39. The heterogenic pool of claim 30, wherein the genome edit is introduced into a gene involved in natural transformation.

40. The heterogenic pool of claim 30, wherein the heterogenic pool comprises all combinations of the two or more co-transformed nucleic acid molecules.

41. An apparatus for introducing two or more populations of nucleic acid molecules into a population of cells in parallel comprising:
a receptacle containing one or more naturally competent cells, wherein the receptacle is configured to produce static conditions that induce natural competence;
a container comprising the two or more populations of nucleic acid molecules, wherein the container is fluidically coupled to the receptacle to introduce the two or more populations of nucleic acid molecules into the receptacle for co-transformation into the naturally competent cells; and
a container comprising selective growth media to replace the natural competence conditions with selective growth media to select co-transformed cells.

42. (canceled)


SEQUENCE LISTING (110 sequences)

SEQ ID NO: 1 - DNA, 24 bp, Artificial Sequence
  Description of Artificial Sequence Synthetic primer
gtagaataagtgcggcgttgagcc

SEQ ID NO: 2 - DNA, 26 bp, Artificial Sequence
  Description of Artificial Sequence Synthetic primer
gattagcaacgattctagcgcaggag

SEQ ID NO: 3 - DNA, 28 bp, Artificial Sequence
  Description of Artificial Sequence Synthetic primer
atttttcagttggcctacaatgctttcc

SEQ ID NO: 4 - DNA, 54 bp, Artificial Sequence
  Description of Artificial Sequence Synthetic primer
gtcgacggatccccggaatacgtttcattagtcacctctattgttaacttgttc

SEQ ID NO: 5 - DNA, 50 bp, Artificial Sequence
  Description of Artificial Sequence Synthetic primer
gaagcagctccagcctacatagtcgaaaataaaaaaaagaggctcgcctc

SEQ ID NO: 6 - DNA, 28 bp, Artificial Sequence
  Description of Artificial Sequence Synthetic primer
cgatgaggataaaaaacacgtacaggcc

SEQ ID NO: 7 - DNA, 25 bp, Artificial Sequence
  Description of Artificial Sequence Synthetic primer
cttgctaaccgttggtgttaccagc

SEQ ID NO: 8 - DNA, 24 bp, Artificial Sequence
  Description of Artificial Sequence Synthetic primer
gcgcccaatgttgtccctttgatg

SEQ ID NO: 9 - DNA, 19 bp, Artificial Sequence
  Description of Artificial Sequence Synthetic primer
attccggggatccgtcgac

SEQ ID NO: 10 - DNA, 19 bp, Artificial Sequence
  Description of Artificial Sequence Synthetic primer
tgtaggctggagctgcttc

SEQ ID NO: 11 - DNA, 22 bp, Artificial Sequence
  Description of Artificial Sequence Synthetic primer
gcgctgccaagtaggtgtaacg

SEQ ID NO: 12 - DNA, 51 bp, Artificial Sequence
  Description of Artificial Sequence Synthetic primer
gtcgacggatccccggaatcataatcttatgtcgctgcttatcatcatctg

SEQ ID NO: 13 - DNA, 50 bp, Artificial Sequence
  Description of Artificial Sequence Synthetic primer
gaagcagctccagcctacatagttattgcccatatctcaagcatggaatc

SEQ ID NO: 14 - DNA, 26 bp, Artificial Sequence
  Description of Artificial Sequence Synthetic primer
agatcttgcctgatgacgctttactc

SEQ ID NO: 15 - DNA, 27 bp, Artificial Sequence
  Description of Artificial Sequence Synthetic primer
accctaagcggttcaattttgtcgatg

SEQ ID NO: 16 - DNA, 23 bp, Artificial Sequence
  Description of Artificial Sequence Synthetic primer
agtgctccggactctttgctctg

SEQ ID NO: 17 - DNA, 26 bp, Artificial Sequence
  Description of Artificial Sequence Synthetic primer
ggctgaacgtggttgtcgaaaatgac

SEQ ID NO: 18 - DNA, 23 bp, Artificial Sequence
  Description of Artificial Sequence Synthetic primer
atcacatgccccaattcgggatg

SEQ ID NO: 19 - DNA, 23 bp, Artificial Sequence
  Description of Artificial Sequence Synthetic primer
cactgctcactagcgatgcagtg

SEQ ID NO: 20 - DNA, 25 bp, Artificial Sequence
  Description of Artificial Sequence Synthetic primer
tgatccgatgatcttttcgcccacc

SEQ ID NO: 21 - DNA, 55 bp, Artificial Sequence
  Description of Artificial Sequence Synthetic primer
gaacatggggtgtacggcagtgccattaaacgatgtgcgggttttgccaatcttg

SEQ ID NO: 22 - DNA, 55 bp, Artificial Sequence
  Description of Artificial Sequence Synthetic primer
caagattggcaaaacccgcacatcgtttaatggcactgccgtacaccccatgttc

SEQ ID NO: 23 - DNA, 55 bp, Artificial Sequence
  Description of Artificial Sequence Synthetic primer
gaacatggggtgtacggcagtgctatttaacgatgtgcgggttttgccaatcttg

SEQ ID NO: 24 - DNA, 55 bp, Artificial Sequence
  Description of Artificial Sequence Synthetic primer
caagattggcaaaacccgcacatcgttaaatagcactgccgtacaccccatgttc

SEQ ID NO: 25 - DNA, 60 bp, Artificial Sequence
  Description of Artificial Sequence Synthetic primer
cctctctccctatagtgagtcgtattaatttcatttaacgatgtgcgggttttgccaatc

SEQ ID NO: 26 - DNA, 57 bp, Artificial Sequence
  Description of Artificial Sequence Synthetic primer
cgttaaatgaaattaatacgactcactatagggagagaggctcgtttggatgttggg

SEQ ID NO: 27 - DNA, 60 bp, Artificial Sequence
  Description of Artificial Sequence Synthetic primer
cgttaaatgaaattaatacgactcactatagggagagaggtttgctctgtttgagaagcc

SEQ ID NO: 28 - DNA, 60 bp, Artificial Sequence
  Description of Artificial Sequence Synthetic primer
atcgttaaatgaaattaatacgactcactatagggagagagggtgaaaacctgttggtgg

SEQ ID NO: 29 - DNA, 60 bp, Artificial Sequence
  Description of Artificial Sequence Synthetic primer
catcgttaaatgaaattaatacgactcactatagggagagaggccaatctcgaaacccac

SEQ ID NO: 30 - DNA, 20 bp, Artificial Sequence
  Description of Artificial Sequence Synthetic primer
gccaatcgtggagtcgaccc

SEQ ID NO: 31 - DNA, 36 bp, Artificial Sequence
  Description of Artificial Sequence Synthetic primer
aaataaaatcaattatcatttgtgttattttagagc

SEQ ID NO: 32 - DNA, 43 bp, Artificial Sequence
  Description of Artificial Sequence Synthetic primer
aaatgataattgattttatttagatttgtgttagagtggcgac

SEQ ID NO: 33 - DNA, 24 bp, Artificial Sequence
  Description of Artificial Sequence Synthetic primer
cgtcacctgaagattccatccgtc

SEQ ID NO: 34 - DNA, 46 bp, Artificial Sequence
  Description of Artificial Sequence Synthetic primer
ggcactgccgtacaccccatgttccggtacccgacaccatcgaatg

SEQ ID NO: 35 - DNA, 44 bp, Artificial Sequence
  Description of Artificial Sequence Synthetic primer
ggctctctggcttattgtggggggaaacctgtcgtgccagctgc

SEQ ID NO: 36 - DNA, 52 bp, Artificial Sequence
  Description of Artificial Sequence Synthetic primer
ttaattgcgttgcgctcactgcccgactcccgttctggataatgttttttgc

SEQ ID NO: 37 - DNA, 25 bp, Artificial Sequence
  Description of Artificial Sequence Synthetic primer
ctgatgaatcccctaatgattttgg

SEQ ID NO: 38 - DNA, 24 bp, Artificial Sequence
  Description of Artificial Sequence Synthetic primer
gtgtggcttgtcggtcggcaaagg

SEQ ID NO: 39 - DNA, 51 bp, Artificial Sequence
  Description of Artificial Sequence Synthetic primer
cgggcagtgagcgcaacgcaattaaatgcaatacttttgcgccagattatg

SEQ ID NO: 40 - DNA, 59 bp, Artificial Sequence
  Description of Artificial Sequence Synthetic primer
caaaatcattaggggattcatcagggggaacgtgattaaaggaksaatggatatgaatg

SEQ ID NO: 41 - DNA, 24 bp, Artificial Sequence
  Description of Artificial Sequence Synthetic primer
gttagaagagcttatccatcaccg

SEQ ID NO: 42 - DNA, 23 bp, Artificial Sequence
  Description of Artificial Sequence Synthetic primer
aaatggagtttgatcgcattggc

SEQ ID NO: 43 - DNA, 49 bp, Artificial Sequence
  Description of Artificial Sequence Synthetic primer
cgggcagtgagcgcaacgcaattaacccctgtatagaaaaaagtttggc

SEQ ID NO: 44 - DNA, 60 bp, Artificial Sequence
  Description of Artificial Sequence Synthetic primer
caaaatcattaggggattcatcaggtgactatccsgagrgagtaatggacgagaataaac

SEQ ID NO: 45 - DNA, 23 bp, Artificial Sequence
  Description of Artificial Sequence Synthetic primer
tgatcagcgtttggaatacgtcg

SEQ ID NO: 46 - DNA, 24 bp, Artificial Sequence
  Description of Artificial Sequence Synthetic primer
aaagcaccaaataatcccgcgacc

SEQ ID NO: 47 - DNA, 47 bp, Artificial Sequence
  Description of Artificial Sequence Synthetic primer
cgggcagtgagcgcaacgcaattaactctgttgggtgaaaaatgtgc

SEQ ID NO: 48 - DNA, 60 bp, Artificial Sequence
  Description of Artificial Sequence Synthetic primer
caaaatcattaggggattcatcagattggcaagsakatacccctatggacgcatcaatcg

SEQ ID NO: 49 - DNA, 23 bp, Artificial Sequence
  Description of Artificial Sequence Synthetic primer
ggcctgcttagcaaatttaccag

SEQ ID NO: 50 - DNA, 24 bp, Artificial Sequence
  Description of Artificial Sequence Synthetic primer
cgctcttatctgcttggataatgg

SEQ ID NO: 51 - DNA, 55 bp, Artificial Sequence
  Description of Artificial Sequence Synthetic primer
gccgctaaatcctgatctttcattaactkscatcatcaaccaaatcgatttgttc

SEQ ID NO: 52 - DNA, 28 bp, Artificial Sequence
  Description of Artificial Sequence Synthetic primer
agttaatgaaagatcaggatttagcggc

SEQ ID NO: 53 - DNA, 23 bp, Artificial Sequence
  Description of Artificial Sequence Synthetic primer
tgaagtacaaggccagttactgg

SEQ ID NO: 54 - DNA, 23 bp, Artificial Sequence
  Description of Artificial Sequence Synthetic primer
tttagccccattggcgaactggg

SEQ ID NO: 55 - DNA, 60 bp, Artificial Sequence
  Description of Artificial Sequence Synthetic primer
gagtatctttgacgtattggatctcatattatactacataatcttatgtcgctgcttatc

SEQ ID NO: 56 - DNA, 60 bp, Artificial Sequence
  Description of Artificial Sequence Synthetic primer
gataagcagcgacataagattatgtagtataatatgagatccaatacgtcaaagatactc

SEQ ID NO: 57 - DNA, 20 bp, Artificial Sequence
  Description of Artificial Sequence Synthetic primer
cggtatcgataagcttgatg

SEQ ID NO: 58 - DNA, 27 bp, Artificial Sequence
  Description of Artificial Sequence Synthetic primer
tggagctgtaatataaaaaccttcttc

SEQ ID NO: 59 - DNA, 30 bp, Artificial Sequence
  Description of Artificial Sequence Synthetic primer
ggagaacttaaatgaattgtagaggacatg

SEQ ID NO: 60 - DNA, 38 bp, Artificial Sequence
  Description of Artificial Sequence Synthetic primer
catcaagcttatcgataccgttccattaggccaccatc

SEQ ID NO: 61 - DNA, 47 bp, Artificial Sequence
  Description of Artificial Sequence Synthetic primer
gaagaaggtttttatattacagctccagggggaaaagagacttaatg

SEQ ID NO: 62 - DNA, 24 bp, Artificial Sequence
  Description of Artificial Sequence Synthetic primer
ctatgtgttcagctgactcccatg

SEQ ID NO: 63 - DNA, 22 bp, Artificial Sequence
  Description of Artificial Sequence Synthetic primer
aaaagtgcacgacaaatagccc

SEQ ID NO: 64 - DNA, 49 bp, Artificial Sequence
  Description of Artificial Sequence Synthetic primer
gacaatgtctgaatcctacaactgctaattcggatctttcatgaggagc

SEQ ID NO: 65 - DNA, 49 bp, Artificial Sequence
  Description of Artificial Sequence Synthetic primer
gctcctcatgaaagatccgaattagcagttgtaggattcagacattgtc

SEQ ID NO: 66 - DNA, 56 bp, Artificial Sequence
  Description of Artificial Sequence Synthetic primer
gacaatatcagcgtcttaaagttgctagtttggatccttcatcaagagttcttcac

SEQ ID NO: 67 - DNA, 56 bp, Artificial Sequence
  Description of Artificial Sequence Synthetic primer
gtgaagaactcttgatgaaggatccaaactagcaactttaagacgctgatattgtc

SEQ ID NO: 68 - DNA, 25 bp, Artificial Sequence
  Description of Artificial Sequence Synthetic primer
aaaggataacaacagatccagctgc

SEQ ID NO: 69 - DNA, 52 bp, Artificial Sequence
  Description of Artificial Sequence Synthetic primer
gttaatgaggtcaagggtggataggttatctaggtagatggaaaatactatg

SEQ ID NO: 70 - DNA, 26 bp, Artificial Sequence
  Description of Artificial Sequence Synthetic primer
tgcataaagcaatccctttctgattt

SEQ ID NO: 71 - DNA, 27 bp, Artificial Sequence
  Description of Artificial Sequence Synthetic primer
gagacattattactgtccaagaaacgc

SEQ ID NO: 72 - DNA, 52 bp, Artificial Sequence
  Description of Artificial Sequence Synthetic primer
catagtattttccatctacctagataacctatccacccttgacctcattaac

SEQ ID NO: 73 - DNA, 25 bp, Artificial Sequence
  Description of Artificial Sequence Synthetic primer
gtgtgggcactcgacatatgacaag

SEQ ID NO: 74 - DNA, 23 bp, Artificial Sequence
  Description of Artificial Sequence Synthetic primer
ctgaatcgagcgttcaataatcg

SEQ ID NO: 75 - DNA, 24 bp, Artificial Sequence
  Description of Artificial Sequence Synthetic primer
cataaacatgtttctgatcagcag

SEQ ID NO: 76 - DNA, 21 bp, Artificial Sequence
  Description of Artificial Sequence Synthetic primer
ttcagggccgaagatctcaac

SEQ ID NO: 77 - DNA, 18 bp, Artificial Sequence
  Description of Artificial Sequence Synthetic primer
gatttggttgatgatgta

SEQ ID NO: 78 - DNA, 18 bp, Artificial Sequence
  Description of Artificial Sequence Synthetic primer
cgatttggttgatgatag

SEQ ID NO: 79 - DNA, 24 bp, Artificial Sequence
  Description of Artificial Sequence Synthetic primer
gccgcttgagtggttaaaaacacc

SEQ ID NO: 80 - DNA, 28 bp, Artificial Sequence
  Description of Artificial Sequence Synthetic primer
gcgacataagattatgtagtataatatg

SEQ ID NO: 81 - DNA, 20 bp, Artificial Sequence
  Description of Artificial Sequence Synthetic primer
agggtatcaatgccgtgacg

SEQ ID NO: 82 - DNA, 20 bp, Artificial Sequence
  Description of Artificial Sequence Synthetic primer
agatccgaattagcagttat

SEQ ID NO: 83 - DNA, 21 bp, Artificial Sequence
  Description of Artificial Sequence Synthetic primer
agctggttgaggatttggtgc

SEQ ID NO: 84 - DNA, 20 bp, Artificial Sequence
  Description of Artificial Sequence Synthetic primer
gcattatctgctcttgagtt

SEQ ID NO: 85 - DNA, 19 bp, Artificial Sequence
  Description of Artificial Sequence Synthetic primer
gatccaaactagcaactct

SEQ ID NO: 86 - DNA, 20 bp, Artificial Sequence
  Description of Artificial Sequence Synthetic primer
ggcagaaagcttgctgtaag

SEQ ID NO: 87 - DNA, 21 bp, Artificial Sequence
  Description of Artificial Sequence Synthetic primer
tcaagggtggataggttattt

SEQ ID NO: 88 - DNA, 19 bp, Artificial Sequence
  Description of Artificial Sequence Synthetic primer
ttttgtcggcgataggttc

SEQ ID NO: 89 - DNA, 43 bp, Artificial Sequence
  Description of Artificial Sequence Synthetic primer
cttatgcgcaacttctccgatattcttcttagccaagattggc

SEQ ID NO: 90 - DNA, 57 bp, Artificial Sequence
  Description of Artificial Sequence Synthetic primer
gtgactggagttcagacgtgtgctcttccgatctccttgcatctgccagttactcgg

SEQ ID NO: 91 - DNA, 68 bp, Artificial Sequence
  Description of Artificial Sequence Synthetic primer
aatgatacggcgaccaccgagatctacactctttcttatgcgcaacttctccgatattcttcttagcc

SEQ ID NO: 92 - DNA, 64 bp, Artificial Sequence
  Description of Artificial Sequence Synthetic primer
caagcagaagacggcatacgagatnnnnnngtgactggagttcagacgtgtgctcttccgatct

SEQ ID NO: 93 - DNA, 28 bp, Artificial Sequence
  Description of Artificial Sequence Synthetic oligonucleotide
cactgccgtacaccccatgttcctttgc

SEQ ID NO: 94 - DNA, 16 bp, Artificial Sequence
  Description of Artificial Sequence Synthetic oligonucleotide
ccccatgttcctttgc

SEQ ID NO: 95 - DNA, 69 bp, Artificial Sequence
  Description of Artificial Sequence Synthetic oligonucleotide
gccaagattggcaaaacccgcacatcgttnnnnnncactgccgtacaccccatgttcctttgcacagtt

SEQ ID NO: 96 - DNA, 69 bp, Artificial Sequence
  Description of Artificial Sequence Synthetic oligonucleotide
gccaagattggcaaaacnnnnnnnnnnnnnnnnnnnnnnnnnnnnnnccccatgttcctttgcacagtt

SEQ ID NO: 97 - DNA, 50 bp, Artificial Sequence
  Description of Artificial Sequence Synthetic oligonucleotide
cggggaacgtgattaaaggaksaatggatatgaatgagcaacagtttttc

SEQ ID NO: 98 - protein, 7 aa, Artificial Sequence
  Description of Artificial Sequence Synthetic peptide
MNEQQFF

SEQ ID NO: 99 - DNA, 50 bp, Artificial Sequence
  Description of Artificial Sequence Synthetic oligonucleotide
cacctgagagtgactatccsgagrgagtaatggacgagaataaacagaag

SEQ ID NO: 100 - protein, 7 aa, Artificial Sequence
  Description of Artificial Sequence Synthetic peptide
MDENKQK

SEQ ID NO: 101 - DNA, 50 bp, Artificial Sequence
  Description of Artificial Sequence Synthetic oligonucleotide
aacaactcaattggcaagsakatacccctatggacgcatcaatcgaaaaa

SEQ ID NO: 102 - protein, 7 aa, Artificial Sequence
  Description of Artificial Sequence Synthetic peptide
MDASIEK

SEQ ID NO: 103 - DNA, 50 bp, Artificial Sequence
  Description of Artificial Sequence Synthetic oligonucleotide
caaatcgatttggttgatgatgsmagttaatgaaagatcaggatttagcg

SEQ ID NO: 104 - protein, 7 aa, Artificial Sequence
  Description of Artificial Sequence Synthetic peptide
MKDQDLA

SEQ ID NO: 105 - DNA, 24 bp, Artificial Sequence
  Description of Artificial Sequence Synthetic oligonucleotide
cacatcgtttaatggcactgccgt

SEQ ID NO: 106 - protein, 4 aa, Artificial Sequence
  Description of Artificial Sequence Synthetic peptide
WHCR

SEQ ID NO: 107 - DNA, 24 bp, Artificial Sequence
  Description of Artificial Sequence Synthetic oligonucleotide
cacatcgttaaatagcactgccgt

SEQ ID NO: 108 - protein, 4 aa, Artificial Sequence
  Description of Artificial Sequence Synthetic peptide
HIVK

SEQ ID NO: 109 - DNA, 24 bp, Vibrio cholerae
  CDS (1)..(24)
cacatcgttaaatggcactgccgt

SEQ ID NO: 110 - protein, 8 aa, Vibrio cholerae
HIVKWHCR